(19) United States
(12) Patent Application Publication
(10) Pub. No.: US 2013/0164753 A1
(43) Pub. Date: Jun. 27, 2013

(54) DIAGNOSTIC METHODS FOR COLITIS

(71) Applicants: John Clare Samuel Harding, Humboldt (CA); Janet Elizabeth Hill, Saskatoon (CA); Manuel Chirino, Saskatoon (CA); Joseph Rubin, Saskatoon (CA)

(72) Inventors: John Clare Samuel Harding, Humboldt (CA); Janet Elizabeth Hill, Saskatoon (CA); Manuel Chirino, Saskatoon (CA); Joseph Rubin, Saskatoon (CA)

(21) Appl. No.: 13/810,567

(22) PCT Filed: Jul. 18, 2011
(86) PCT No.: PCT/CA2011/000828

§ 371 Date: Jan. 16, 2013

Related U.S. Application Data
(60) Provisional application No. 61/365,140, filed on Jul. 16, 2010.

(51) Int. Cl. C12Q 1/68; G01N 33/569

(52) U.S. Cl. CPC C12Q 1/689; G01N 33/56911
USPC 435/6.11; 435/6.12; 435/7.1; 435/7.92; 435/7.4; 536/24.33; 435/189; 530/350; 530/300; 530/387.9; 435/287.2

(57) ABSTRACT

A method of screening for, diagnosing or detecting the presence of Brachyspira sp. Sask30446 and/or colitis in a subject comprising detecting a Brachyspira sp. Sask30446 polynucleotide or polypeptide in a sample of the subject, wherein detection of the Brachyspira sp. Sask30446 polynucleotide or polypeptide is indicative of the presence Brachyspira sp. Sask30446 and/or that the subject has colitis or has an increased risk of developing colitis.

This application claims the benefit of U.S. Provisional Application No. 61/365,140, filed Jul. 16, 2010, which is incorporated herein in its entirety by reference.

### FIELD OF THE DISCLOSURE

The present disclosure relates to methods and compositions for detecting colitis, and particularly to methods and compositions for detecting a mucohemorhagic colitis associated with a Brachyspira species that is not B. pilosicoli or B. hyodyseneriae.

### BACKGROUND OF THE DISCLOSURE

Five Brachyspira spp. commonly infect pigs 3 months of age and older. B. hyodysenteriae (Bh) and B. pilosicoli (Bp) cause swine dysentery and spirochetal colitis respectively, whereas B. innocens (Binn), and B. intermedia (Bint) are generally considered avirulent. B. murdochii (Bm) has only recently been associated with catarrhal colitis (Jansen et al, 2010).

Atypical Brachyspira spp. associated with colitis have been reported (Thompson et al, 1998; Thompson et at 2001). However no detailed genomic information on the specific strains has been provided.

### SUMMARY OF THE DISCLOSURE

An aspect includes a method of screening for or detecting a presence of Brachyspira sp. Sask30446 organism in a sample from a subject comprising determining a level of Brachyspira sp. Sask30446 polynucleotide or polypeptide in the sample.

In an embodiment, the determining step comprises quantitating the level of Brachyspira sp. Sask30446 polynucleotide or polypeptide in the sample.

Determining the presence or an increased level of Brachyspira sp. Sask 30446 organism in a sample from a subject can be indicative the subject has a Brachyspira species infection related to Brachyspira sp. Sask30446 and/or unrelated to B. hyodysenteriae (Bh) and/or B. pilosicoli (Bp).

Accordingly, another aspect includes a method of detecting or diagnosing a Brachyspira species infection related to Brachyspira sp. Sask30446 and/or unrelated to B. hyodysenteriae (Bh) and/or B. pilosicoli (Bp) in a subject comprising detecting a level of Brachyspira sp. Sask30446 polynucleotide or polypeptide in the sample. In an embodiment, the method further comprises confirming that the subject is free of a B. hyodysenteriae (Bh) and/or B. pilosicoli (Bp) infection. In an embodiment, the method is for detecting or diagnosing a Brachyspira species infection related to Brachyspira sp. Sask30446 and/or unrelated to B. hyodysenteriae (Bh), B. pilosicoli (Bp), B. innocens (Binn), B. intermedia (Bint) and/or B. murdochii (Bm) in a subject comprising determining a level of Brachyspira sp. Sask30446 polynucleotide and/or polypeptide in the sample. In a further embodiment, the method further comprises confirming that the subject is free of a B. hyodysenteriae (Bh), B. pilosicoli (Bp), B. innocens (Binn), B. intermedia (Bint) and/or B. murdochii (Bm) infection.

It is demonstrated that the presence or level of Brachyspira sp. Sask30446 is associated with colitis related to Brachyspira sp. Sask30446 and/or unrelated to B. hyodysenteriae (Bh) and/or B. pilosicoli (Bp).

Accordingly, a further aspect includes a method of screening for, diagnosing or detecting colitis related to Brachyspira sp. Sask30446 and/or unrelated to B. hyodysenteriae and/or B. pilosicoli in a subject comprising determining a level of a Brachyspira sp. Sask30446 polynucleotide or polypeptide in a sample of the subject such as a colon tissue sample, wherein detecting the Brachyspira sp. Sask30446 polynucleotide or polypeptide is indicative that the subject has colitis related to Brachyspira sp. Sask30446 and/or unrelated to B. hyodysenteriae and/or B. pilosicoli or has an increased risk of developing colitis related to Brachyspira sp. Sask30446 and/or unrelated to B. hyodysenteriae and/or B. pilosicoli.

In an embodiment, detection of an increased level of the Brachyspira sp. Sask30446 polynucleotide or polypeptide compared to a control diagnoses a Brachyspira infection related to Brachyspira sp. Sask30446 and/or unrelated to B. hyodysenteriae (Bh) and/or B. pilosicoli (Bp) and/or is indicative the subject has colitis related to Brachyspira sp. Sask30446 and/or unrelated to B. hyodysenteriae and/or B. pilosicoli or has an increased risk of developing colitis related to Brachyspira sp. Sask30446 and/or unrelated to B. hyodysenteriae and/or B. pilosicoli.

In an embodiment, the Brachyspira sp. Sask30446 polynucleotide or polypeptide is selected from NADPH oxidase (nox1), chaperonin 60 (cpn60), esterase (est), glucose kinase (glpk), phosphglucomutase (pgm) acetyl-CoA acetyltransferase (thi) and small subunit ribosomal RNA (16S rRNA).

In an embodiment, detection of—and/or detection of an increased level of a NADPH oxidase polynucleotide with at least 92.3% identity to any one of SEQ ID NOs: 7 to 9 or a NADPH polypeptide with at least 92.3% identity to any one of SEQ ID NOs: 11 and 12 is indicative for the presence of the Brachyspira sp. Sask30446 organism.

In an embodiment, detection of—and/or detection of an increased level of a cpn60 polynucleotide with at least 97.5% identity to SEQ ID NO: 25 or a cpn60 polypeptide with at least 98.5% identity to SEQ ID NO: 26 is indicative for the presence of the Brachyspira sp. Sask30446 organism.

In an embodiment, detection of—and/or detection of an increased level of an est polynucleotide with at least 93.5% identity to SEQ ID NO: 27 or an est polypeptide with at least 95.5% identity to SEQ ID NO: 28 is indicative for the presence of the Brachyspira sp. Sask30446 organism.

In an embodiment, detection of—and/or detection of an increased level of a glpk polynucleotide with at least 95.5% sequence identity to SEQ ID NO: 29 or a glpk polypeptide with at least 99.5% sequence identity to SEQ ID NO: 30 is indicative for the presence of the Brachyspira sp. Sask30446 organism.

In an embodiment, detection of—and/or detection of an increased level of a pgm polynucleotide with at least 93.5% sequence identity to SEQ ID NO: 31 or a pgm polypeptide with at least 99.5% sequence identity to SEQ ID NO: 32 is indicative for the presence of the Brachyspira sp. Sask30446 organism.

In an embodiment, detection of—and/or detection of an increased level of a thi polynucleotide with at least 92.5% sequence identity to SEQ ID NO: 33 or a thi polypeptide with at least 95.5% sequence identity to SEQ ID NO: 34 is indicative for the presence of the Brachyspira sp. Sask30446 organism.

In an embodiment, detection of a 16S rRNA polynucleotide with at least 99.5% sequence identity to SEQ ID NO: 37 is indicative for the presence of the Brachyspira sp. Sask30446 organism.

As mentioned, the presence or increased level of Brachyspira sp. Sask30446 is associated with a Brachyspira infection related to Brachyspira sp. Sask30446 and/or unrelated to B. hyodysenteriae (Bh) and/or B. pilosicoli (Bp) and/or colitis related to Brachyspira sp. Sask30446 and/or unrelated to B. hyodysenteriae (Bh) and/or B. pilosicoli (Bp).

In an embodiment, the level of the Brachyspira sp. Sask30446 polynucleotide or polypeptide is increased at least 2 fold, at least 3 fold, at least 4 fold, at least 5 fold, at least 6 fold, at least 7 fold, at least 8 fold, at least 9 fold or at least 10 fold.

In an embodiment, the colitis is a mucohaemorrhagic colitis associated with bloody diarrhea.

In an embodiment, the sample comprises cells or tissue of stomach, duodenum, ileum, jejunum, caecum, rectum or colon, for example colon cells, or fecal material and/or gastrointestinal content such as colon contents. In an embodiment, the sample comprises cells or tissue of, caecum, rectum or colon, for example colon cells, or fecal material and/or gastrointestinal content such as colon contents.

In an embodiment, the sample comprises colonic cells. In an embodiment, the sample comprises colon contents. In another embodiment, the sample is a fecal sample and/or comprises fecal material.

In another embodiment, the subject is a pig.

Another aspect includes, an isolated polynucleotide comprising at least 15 contiguous nucleotides of anyone of SEQ ID NOs: 3-9, 25, 27, 29, 31, 33 and 37.

A further aspect includes an isolated polypeptide comprising at least 10 contiguous amino acids of SEQ ID NO:11, 12, 26, 28, 30, 32 and 34 or encoded by any one of SEQ ID NOs: 7 to 9, 25, 27, 29, 31, 33 and 37.

Another aspect includes an isolated antibody specific for the isolated polypeptide described herein.

Also provided are composition and kits comprising the isolated polynucleotide, the isolated polypeptide or the isolated antibody described herein, and optionally a suitable carrier. In an embodiment the kit comprises one or more additional components selected from a polynucleotide useful as a positive or negative control; an analyte specific binding agent specific for a Brachyspira sp. Sask30446 polynucleotide or polypeptide; a standard and a reaction tube or plate.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be discussed in relation to the drawings in which:

FIG. 1. Intestinal contents (A) and colonic mucosa (B, C, D) of pigs without (B) or with mucohaemorrhagic colitis (C, D).

FIG. 2. Phylogenetic trees. A) Phylogenetic tree of 810 bp nox1 gene sequence from colitis cases and published reference sequences; B) Phylogenetic relationships of Brachyspira spp. based on partial nox1 peptide sequences.

FIG. 3A. qPCR detection of Brachyspira sp. Sask30446 in intestinal tissue and contents of affected and unaffected pigs in affected barn (Farm D). Pigs D1008030 and D1008033 are unaffected pigs; Pigs D1008031 and D1008032 are affected.

FIG. 3B. qPCR detection of Brachyspira sp. Sask30446 in intestinal tissue and contents of affected and unaffected pigs in affected barn (Farm L). Pigs D1100925 and D1100928 are unaffected pigs; Pigs D1100927, D1100929 and D1100930 are affected pigs.

FIG. 4. Bootstrapped phylogenetic tree based on the 555 bp cpn60 universal target from representative Brachypira spp., including Sask30446.

FIG. 5. Growth of Brachyspira sp. Sask30446 on BAM-SR agar after 8 days at 37° C. (top). Colonies are <1 mm in diameter and have a “fried egg” shape. Weak haemolysis is apparent.

FIG. 6. Gram stain of colony from plate in FIG. 5. Lower panel shows detail of slide with 5.0 μm scale bar.

FIG. 7. Gram stain of colon contents from pig with hemorrhagic colitis showing abundant spirochetes (upper) and detail of same slide (lower). qPCR results using primers JH0224/JH0225 indicated ˜1×10⁷ Sask30446 organisms per gram of feces.

### DETAILED DESCRIPTION OF THE DISCLOSURE

A novel Brachyspira sp. associated with haemorrhagic colitis in pigs has been detected (herein referred to as Brachyspira sp. Sask30446). The disease is clinically and pathologically similar to swine dysentery caused by B. hyodysenteriae, but neither this nor other Brachyspira species are evident in affected pigs from the 2 farms investigated. This novel Brachyspira sp. affects pigs from about 3 months old, results primarily in a bloody diarrhea, but will result in death when environmental conditions are inappropriate.

I. Glossary

The term “analyte specific binding agent” or “ASBA” refers to a substance that specifically binds to an analyte. For example, if the analyte is the NADPH oxidase polynucleotide of Brachyspira sp. Sask30446 detected as described herein (e.g. NADPH oxidase polynucleotide), the ASBA may be a probe. In another example, if the analyte is the NADPH oxidase polypeptide of Brachyspira sp. Sask30446 (e.g. Brachyspira sp. Sask30446 NADPH oxidase polypeptide), the ASBA may be an antibody. A substance “specifically binds” a polynucleotide or polypeptide if it reacts at a detectable level with a target analyte polynucleotide or polypeptide and is determinative of the presence of the analyte (e.g. polypeptide or nucleic acid) often in a heterogeneous population of macromolecules and binds materially better and/or with greater affinity to the target analyte polynucleotide or polypeptide compared to a non-target analyte (e.g. any other known analyte), for example binds with significantly higher affinity, for example with at least 3, at least 5, at least 10, at least 20 times or more greater affinity to the target analyte than a non-target analyte. As another example, when the ASBA is a probe, “specifically binds” refers to the specified probe under hybridization conditions binding to a particular gene sequence at least 1.5, at least 2, at least 3, at least 5, at least 10, at least or 20 times background, where background is for example the level of binding detectable with hybridization with hybridization solution alone or with a control probe such as to a house keeping gene or other different polynucleotide sequence. Binding properties may be assessed using DNA hybridization for example when the ASBA is a polynucleotide probe, or by Western blot or with an ELISA when the ASBA is an antibody, methods which may be readily performed by those skilled in the art (see for example, Newton et al., Develop. Dynamics 197: 1-13, 1993). The term “antibody” as used herein is intended to include monoclonal antibodies, polyclonal antibodies, and chimeric antibodies. The antibody may be from recombinant sources and/or produced in transgenic or non-transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

Antibodies may be monospecific, bispecific, trispecific or of greater multispecificity. Multispecific antibodies may immunospecifically bind to different epitopes of a NADPH oxidase polypeptide and/or or a solid support material. Antibodies may be from any animal origin including birds and mammals (e.g., human, murine, donkey, sheep, rabbit, goat, guinea pig, camel, horse, or chicken). Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. In aspects, the antibody is a purified or isolated antibody. By “purified” or “isolated” is meant that a given antibody or fragment thereof, whether it that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

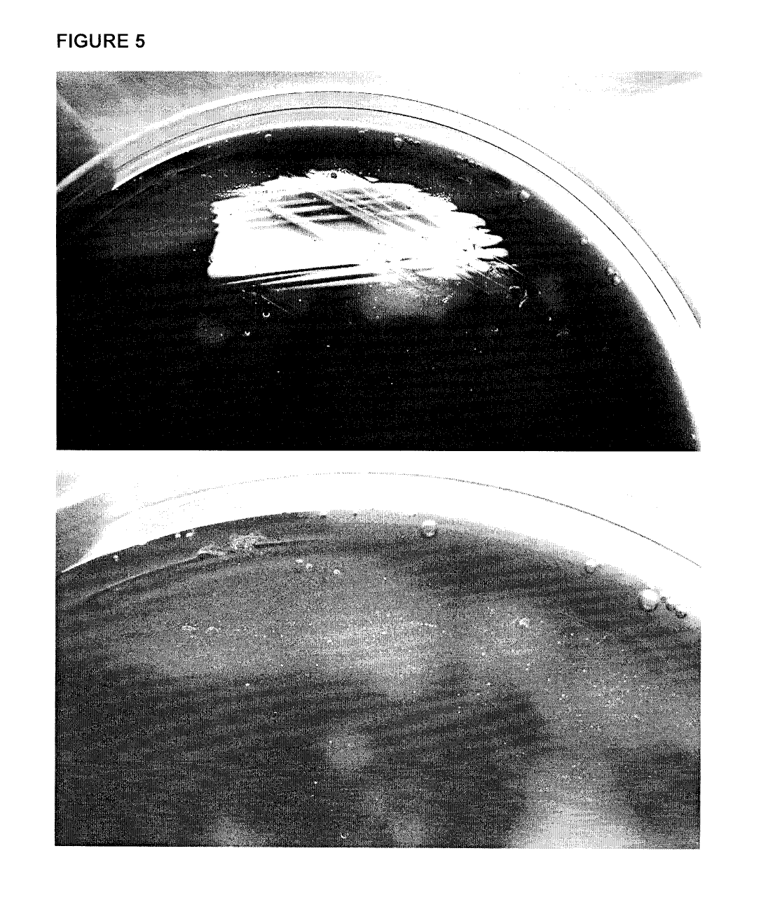

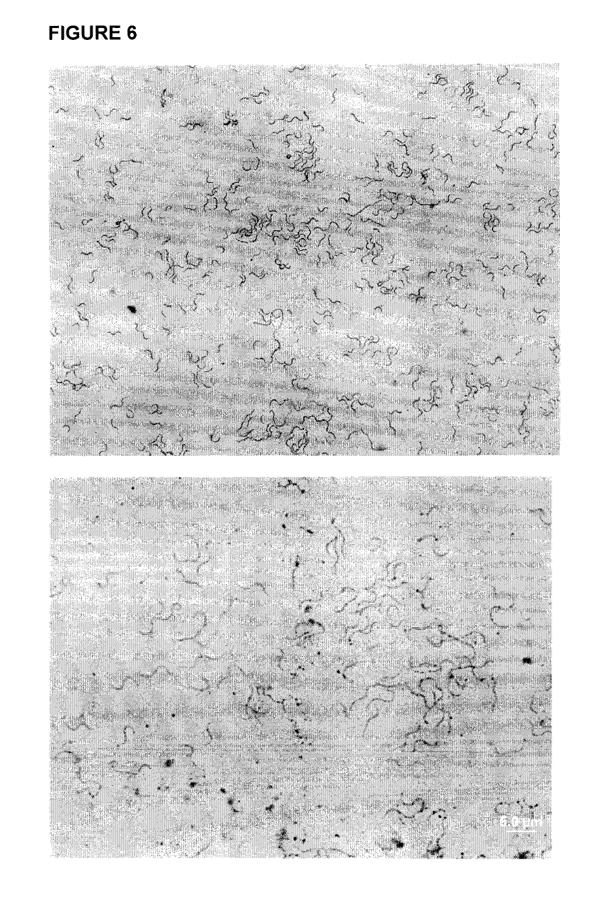

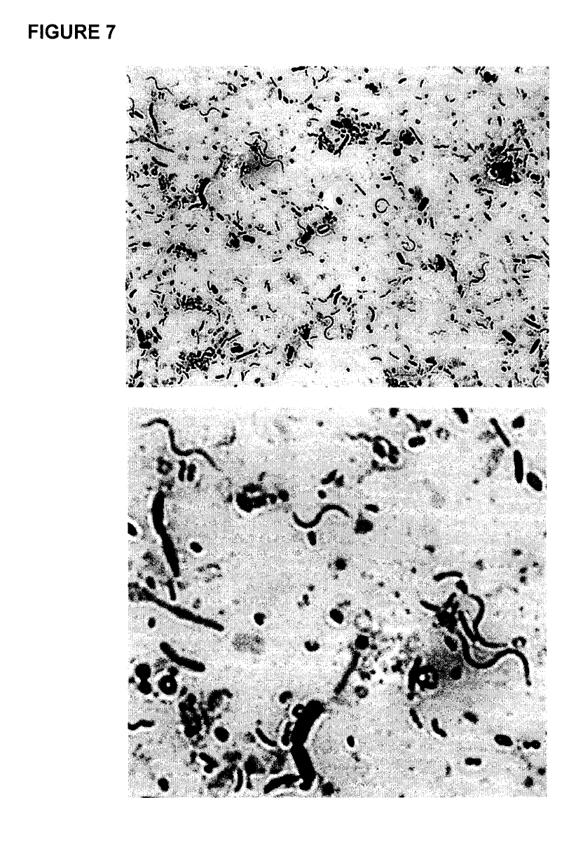

The term “Brachyspira sp. Sask30446” refers to a species of Brachyspira comprising one or more of SEQ ID NOs: 11, 12, 26, 28, 30, 32, and 34 and/or a sequence with at least 92.5, 93.5, 94.5 95.5, 96.5, 97.5, 98.5, 99 or 99.5% identity with a polynucleotide sequence encoding SEQ ID NOs: 11, 12, 26, 28, 30, 32, and/or 34. For example, NADPH oxidase polynucleotides from isolates of Brachyspira sp. Sask30446 were found to share 99% identity at the polynucleotide level (e.g. SEQ ID NOs:7-9), whereas the closest DNA relative shared only 92.2% identity (SEQ ID NO:10). Brachyspira sp. Sask30446 for example, when cultured on BAM-SR agar supplemented with horse blood, under anaerobic conditions at for example 37-44° C., grows as weakly hemolytic, small, clear, wet looking, “fried egg” shaped colonies (as in FIG. 5). Gram stain of isolates from agar plates show pleiomorphic, Gram negative spirochetes with tapered ends. Length of the cells is variable, for example 2-20 μm (FIG. 6). Spirochetes with the same morphology are abundant in samples of colon contents, colon tissue and/or feces from pigs with hemorrhagic colitis that also test positive by the B. sp. Sask30446 qPCR assay (primers JH0224/JH0225) with counts of ˜1×10⁷ B. sp. Sask30446 organisms per gram of feces or colon contents (FIG. 7). In an embodiment, copies of DNA is a proxy for organism number. For example, as organisms typically comprise 1 genomic copy of each gene, it is assumed that the number of DNA copies amplified is reflective of the approximate number of organisms, for example within 2 fold.

The term “colon” as used herein means the large intestine, for example the large intestine of a subject pig, and “colon contents” means digested material contained within the colon including the rectum.

The term “colonic tissue” as used herein means tissue derived from the colon.

The term “colon cell” as used herein refers to a cell of colonic tissue.

The term “caecum” as used herein means the first portion of the large intestine that forms an elongated dilated pouch in the pig.

The term “fecal material” as used herein refers to digestive waste products that have been expelled from the subject during defecation.

The term “composition” includes any composition of matter, including polynucleotides, polypeptides, proteins, mixtures, or antibodies, for example that are useful in the methods described herein.

The term “level” as used herein refers to an absolute or relative quantity of a polynucleotide or polypeptide that is detectable or measurable in a sample comprising a cell, tissue or GI contents, from a subject. The level can be a numerical value and/or range and can refer to polypeptide levels or nucleic acid levels.

The term “expression level” as used herein refers to the absolute or relative amount of the transcription and/or translation product of a gene described herein and includes RNA and polypeptide products. A person skilled in the art will be familiar with a number of methods that can be used to determine RNA transcription levels, such as qRT-PCR and/or polypeptide levels such as immunohistochemistry and/or western blotting.

The term “increased level” or “elevated level” as used herein in refers to a detectable or quantifiable increase, and for example at least a 20%, 30%, 40% or at least 50% increase in the measurable level in a sample as compared with the measurable level in a control or comparator sample of the same type e.g. same tissue or material (e.g. fecal samples).

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, and which can represent the sense or antisense strand. The term also includes modified or substituted oligomers comprising non-naturally occurring monomers or portions thereof, which function similarly, which are referred to herein as “chemical analogues” and/or “oligonucleotide analogues” such as “peptide nucleic acids”. Such modified or substituted nucleic acids may be preferred over naturally occurring forms because of properties such as increased stability in the presence of nucleases.

The term “isolated polynucleotide”, “isolated oligonucleotide” and/or “isolated nucleic acid” as used herein refers to a nucleic acid substantially free of cellular material or to a culture medium when produced by recombinant DNA techniques, or chemical precursors, or other chemicals when chemically synthesized.

The terms “peptide”, “polypeptide” and “protein” are used interchangeably and as used herein refer to more than one amino acid joined by a peptide bond.

The term “isolated polypeptide” as used herein refers to a proteinaceous agent, such as a peptide, polypeptide or protein, which is substantially free of cellular material or culture medium when produced recombinantly, or to chemical precursors, or other chemicals, when chemically synthesized.

The phrase “Brachyspira sp. Sask30446 polynucleotide” as used herein refers to a polynucleotide having for example at least 92.5% sequence identity to any one of SEQ ID NOs: 7 to 9; a cpn-60 polynucleotide having for example at least 97.5% sequence identity to SEQ ID NO:26; an est polynucleotide having for example at least 93.5% sequence identity with SEQ ID NO:27; a glpk polynucleotide having for example at least 95.5% sequence identity with SEQ ID NO: 29; a pgm polynucleotide having for example at least 93.5% sequence identity with SEQ ID NO: 31 a thi polynucleotide having for example at least 92.5% sequence identity with SEQ ID NO:33; a 16S rRNA polynucleotide having for example, at least 99.5% sequence identity with SEQ ID NO:37 and/or any polynucleotide sequence present in a Brachyspira species comprising any one of SEQ ID NOs:7-9, 25, 27, 29, 31, 33, 35 and 37; and/or which is found in a Brachyspira species associated with haemorrhagic colitis in pigs, wherein the Brachyspira species is not B. pilosicoli and/or B. hyodysenteriae and/or B. murdochii and includes native-sequence polynucleotides, and naturally occurring variants including a portion of a polynucleotide, an isoform, precursor, complex, or modified form and derivatives of the polynucleotide. The polynucleotide can have at least 92.5%, 93.5%, 94.5%, 95.5%, 96.5%, 97.5%, 98.5%, 99% or at least 99.5% or more sequence identity with a sequence of SEQ ID NOs: 7 to 9, 25, 27, 29, 31, 33, and 37. For example, Brachyspira sp. Sask30446 NADPH oxidase polynucleotides have been identified which share about 99% sequence identity.

Brachyspira sp. Sask30446 polynucleotides further include sequences that differ from a native sequence due to degeneracy in the genetic code. As one example, DNA sequence polymorphisms within the nucleotide sequence of a Brachyspira sp. Sask30446 NADPH oxidase polynucleotide may result in silent mutations that do not affect the amino acid sequence. Variations in one or more nucleotides may exist among subjects within a population due to natural allelic variation. DNA sequence polymorphisms may also occur which lead to conservative changes in the amino acid sequence of a polypeptide. Fragments are also included.

Brachyspira sp. Sask30446 polynucleotides also include nucleic acids that hybridize under stringent conditions, preferably high stringency conditions to SEQ ID NOs: 7-9, 25, 27, 29, 31, 33, 35, 37, which when expressed retain activity.

Brachyspira sp. Sask30446 polynucleotides include complementary nucleic acid sequences, and nucleic acids that are substantially identical to these sequences (e.g. at least 92.5%, 93%, 94%, 95%, 96%, 97%, 98%, or at least 99% sequence identity).

For example, the phrase “Brachyspira sp. Sask30446 NADPH oxidase polynucleotide” as used herein refers to a NADPH oxidase polynucleotide comprising at least 92.5%, 93.5, 94.5, 95.5, 96.5, 97.5, 98.5, 99, or 99.5 sequence identity to any one of SEQ ID NOs: 7 to 9 and which is found in a Brachyspira species associated with haemorrhagic colitis in pigs, wherein the Brachyspira species is not B. pilosicoli and/or B. hyodysenteriae and/or B. murdochii and includes native-sequence polynucleotides, and naturally occurring variants including a portion of a polynucleotide, an isoform, precursor, complex, or modified form and derivatives of the polynucleotide. The polynucleotide can have at least 93%, 94%, 95%, 96%, 97%, 98% or 99% or more sequence identity with a sequence of SEQ ID NO: 7 to 9. For example, variant Brachyspira sp. Sask30446 NADPH oxidase polynucleotides have been identified which share about 99% sequence identity. Brachyspira sp. Sask30446 NADPH oxidase polynucleotides include complementary nucleic acid sequences, and nucleic acids that are substantially identical to these sequences (e.g. at least about 92.3%, preferably 93%, 94%, 95%, 96%, 97%, 98%, or 99% sequence identity).

Brachyspira sp. Sask30446 NADPH oxidase polynucleotides further include sequences that differ from a native sequence due to degeneracy in the genetic code. As one example, DNA sequence polymorphisms within the nucleotide sequence of a Brachyspira sp. Sask30446 NADPH oxidase polynucleotide may result in silent mutations that do not affect the amino acid sequence. Variations in one or more nucleotides may exist among subjects within a population due to natural allelic variation. DNA sequence polymorphisms may also occur which lead to changes in the amino acid sequence of a polypeptide.

Brachyspira sp. Sask30446 NADPH oxidase polynucleotides also include nucleic acids that hybridize under stringent conditions, preferably high stringency conditions to a Brachyspira sp. Sask30446 NADPH oxidase polynucleotide.

The term “reference sequence” as used herein refers to a Brachyspira spp. sequence for a known species, including for example the sequences provided herein for Brachyspira sp. Sask30446, to which an polynucleotide or polypeptide can be compared to in identifying the species identity of a particular sequence. The reference sequence can for example be comprised in a database.

The term “determining a level” as used herein in respect of determining a level of a Brachyspira sp. Sask30446 polynucleotide or polypeptide such as a Brachyspira sp. Sask30446 nox, cpn60, est, glpk, pgm, thi and 16S rRNA polynucleotide comprises the application of a method to a sample, for ascertaining or measuring quantitatively, semi-quantitatively or qualitatively the amount of the polynucleotide or polypeptide in the sample. For example, the presence of or a level of Brachyspira sp. Sask30446 polynucleotide can be determined by PCR amplification such as quantitative PCR using for example real-time PCR with species-specific primers such as SEQ ID NOs: 3-6. Amplification is indicative of the presence of Brachyspira sp. Sask30446 organism and depending on the PCR protocol used, indicative of the level of polynucleotide, which can be quantified. Alternatively, the presence of or a level of a Brachyspira sp. Sask30446 polynucleotide can be determined by PCR amplification using broad spectrum primers such as SEQ ID NOs:1-2, 15-24 and 35-36 to amplify for example one or more of Brachyspira sp. Sask30446 cpn60, est, glpk, pgm, thi and 16S rRNA followed by sequence analysis and comparison to one or more corresponding reference sequences (e.g. a Brachyspira sp. Sask30446 polynucleotide described herein). Sequence analysis is used to determine if the sequence is most similar to a Brachyspira sp. Sask30446 polynucleotide sequence. A person skilled in the art would understand how to design specific species-specific primers for Brachyspira sp. Sask30446 polynucleotides such as cpn60, est, glpk, pgm, thi and 16S rRNA in light of the sequences provided herein. Quantitative PCR methods such as real-time PCR can be used. For example, the PCR method can be SYBR green real time PCR. SYBR green is a fluorescent dye and intercalating agent that binds double-stranded DNA. During SYBR green real-time PCR, SYBR green dye is added to the PCR reaction and the thermocycler used is equipped with a camera that permits real-time monitoring of fluorescence accumulation during the run (each well or tube is measured at each cycle of the PCR). Real-time PCR assays can for example be more sensitive than conventional, end-point, PCR (e.g. where the products are analysed on an agarose gel after the reaction is completed). Methods that can be employed to detect Brachyspira spp. polynucleotides also include for example, sequence analysis, microarray, in situ hybridization other hybridization based methods and/or a combination thereof. For example, the polynucleotide can be amplified for example by PCR, detecting by hybridization for example by Southern analysis or in situ hybridization. Other techniques include, quantitative real-time PCR, multiplex ligation dependent probe amplification (MLPA), nucleic acid sequence based amplification (NASBA) and/or real time NASBA. As used herein “NASBA” refers to a sensitive isothermal transcription-based amplification method used for example for RNA research. NASBA technology is optionally applied to single nucleotide polymorphism (SNP) analysis using human genomic DNA as a template. For example combination of DNA NASBA with multiplex hybridization of specific molecular beacons makes it possible to discriminate the presence of polynucleotides of interest.

Polypeptide levels can be determined by a number of methods including for example, immunoassays including for example immunohistochemistry, ELISA, e.g. sandwich type ELISA, Western blot, immunoprecipitation, immunofluorescence, radioimmunoassay, dot blotting, FACS and the like, where an ASBA specific detection agent such as an antibody for example, a labeled antibody, specifically binds the Brachyspira sp. Sask30446 polypeptide (e.g. a polypeptide having SEQ ID NOs:24, 26, 28, 30, 32, 34, or 36).

The term “quantitating a level” as used herein means semi-quantitatively and/or quantitatively determining a level of a polynucleotide or polypeptide. For example, as described above, the method can employ quantitative PCR and for example a fluorescence monitor or camera, densitometric methods (e.g. applied to PCR products separated by gel electrophoresis) as well as other methods known in the art.

The term “detectable label” as used herein means an agent or composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins and/or polynucleotides (e.g. probes) which can be made detectable, e.g., by incorporating a radiolabel into the peptide or nucleic acid.

The term “hybridize” refers to the sequence specific non-covalent binding interaction with a complementary nucleic acid.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40, 50 or more) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10[ Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The Brachyspira sp. Sask30446 NADPH oxidase polynucleotides are intended to include DNA and RNA (e.g. mRNA) as well as complementary DNA (i.e. cDNA) and can be either double stranded or single stranded. A polynucleotide may, but need not, include additional coding or non-coding sequences, or it may, but need not, be linked to other molecules and/or carrier or support materials. The polynucleotides for use in the methods disclosed herein may be of any length suitable for a particular method. In certain applications the term refers to antisense polynucleotides (e.g. mRNA or DNA strand in the reverse orientation to sense Brachyspira sp. polynucleotide such as NADPH oxidase polynucleotide).

The term “Brachyspira sp. Sask30446 polypeptide” as used herein refers to a polypeptide comprising at least 92.5%, 93.5%, 94.5%, 96.5%, 96.5%, 97.5%, 98.5%, 99% or at least 99.5% sequence identity to SEQ ID NOs: 11, 12, 26, 28, 30, 32, 24 or 36, a polypeptide encoded by any one of SEQ ID NOs: 7 to 9, 25, 27, 29, 31, 33, 35 or 37, any polypeptide expressed in a Brachyspira species comprising any one of SEQ ID NOs: 11, 12, 26, 28, 30, 32, 24 or 36 and/or of a Brachyspira species associated with haemorrhagic colitis in pigs, wherein the Brachyspira species is not B. pilosicoli and/or B. hyodysenteriae and/or B. murdochii and in particular includes the native-sequence polypeptide, isoforms, all homologs, fragments, precursors, complexes, and modified forms and derivatives thereof. For example, a Brachyspira sp. Sask30446 NADPH oxidase polypeptide sequence comprises 270 amino acids (encoded by 810 nucleotides) and is 92.3% identical (97% similar) to both EF517544 Brachyspira innocens and DQ487124 Brachyspira suanatina.

“Percent identity” of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

The term “primer” as used herein refers to a polynucleotide, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “probe” as used herein refers to a nucleic acid sequence that will hybridize to a nucleic acid target sequence. In one example, the probe hybridizes to a NADPH oxidase DNA (e.g. genomic DNA), in another example the probe hybridizes to a NADPH oxidase RNA or a nucleic acid sequence complementary to the NADPH oxidase RNA. The length of probe depends for example, on the hybridization conditions and the sequences of the probe and nucleic acid target sequence. The probe can be for example, at least 15, 20, 25, 50, 75, 100, 150, 200, 250, 400, 500 or more nucleotides in length.

A person skilled in the art would recognize that “all or part of” of a particular probe or primer can be used as long as the portion is sufficient for example in the case a probe, to specifically hybridize to the intended target and in the case of a primer, sufficient to prime amplification of the intended template.

The terms “sample”, “biological sample”, and the like mean a material known or suspected of containing Brachyspira sp. organisms, polypeptides and/or polynucleotides. A test sample can be used directly as obtained from the source, after culturing for example on an agar plate or following a pretreatment to modify the character of the sample. The sample can be derived from any biological gastrointestinal source, such as tissues, extracts, or cell cultures, including primary cells (e.g. colon cells), cell lysates, as well as the contents of gastrointestinal tissues (e.g. jejunum content) and/or their end products such as feces. For example the sample can comprise contents, cells, and/or tissues derived from stomach, duodenum, ileum, jejunum, colon, caecum and/or rectum. The sample can also be a fixed sample, such as a formalin fixed tissue sample for example for PCR analysis in situ hybridization or immunohistochemical analysis or a fresh tissue such as a biopsy, frozen tissue or paraffin embedded tissue (e.g. fresh or fixed, for example fixed in 10% formalin). For example, the sample can be a fixed sample, for example a fixed tissue block or tissue section adhered to a slide. The sample can be fixed as in tissue, fresh or frozen as in contents or tissue, or preserved in other reagents and compounds, for example glutaraldehyde for electron microscopy evaluation of tissue. The sample can be obtained from animals, preferably mammals, most preferably pigs. The sample can be treated prior to use, such as diluting viscous fluids, and the like. Methods of treatment can involve filtration, distillation, extraction, concentration, inactivation of interfering components, the addition of reagents, and the like. The sample is for example processed to isolate genomic DNA. A number of methods and kits are known in the art for isolating DNA from tissue and stool such as Qiagen DNeasy Blood & Tissue Kit or QIAamp DNA Stool Mini kit for feces.

A “significant increase” in a level of a Brachyspira sp. Sask30446 NADPH oxidase polynucleotide or polypeptide, in a sample compared to a control or standard (e.g. predetermined levels in a standard or levels in other samples from a subject) may represent levels that are higher or lower than the standard error of the detection assay. In particular embodiments, the levels may be at least about 2, 5, 10, 50, 100, 500, 1000 or times higher than the control or standard.

The term “subject” refers to an animal including a warm-blooded animal such as a mammal, for example which is afflicted with or suspected of having or being pre-disposed to a bacterial disease. Mammal includes without limitation any members of the Mammalia. The terms also include animals bred for food, sport, or as pets, including domestic animals such as horses, cows, sheep, poultry, fish, pigs, and goats, and cats, dogs, and zoo animals, non-human primates (e.g. gorilla or chimpanzee), and rodents such as rats and mice.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

II. Methods

In an aspect, the disclosure includes a method of screening for or detecting a presence of a Brachyspira sp. Sask30446 organism in a sample comprising determining a level of Brachyspira sp. Sask30446 polynucleotide or polypeptide in the sample, wherein detection of the Brachyspira sp. Sask30446 polynucleotide or polypeptide in the sample is indicative of the presence of Brachyspira sp. Sask30446 organism in the sample. In an embodiment, the determining step comprises determining a level of Brachyspira sp. Sask30446 NADPH oxidase, cpn60, est, plgk, pgm thi or 16S rRNA polynucleotide.

In an embodiment, detection of a NADPH oxidase polynucleotide with at least 92.5% identity to any one of SEQ ID NOs: 7 to 9 or a NADPH oxidase polypeptide with at least 92.5% identity to SEQ ID NO:11 or 12 is indicative for the presence of the Brachyspira sp. Sask30446 organism.

In an embodiment, detection of a cpn60 polynucleotide with at least 97.5% identity to SEQ ID NO: 25 or a cpn60 polypeptide with at least 98.5% identity to SEQ ID NO: 26 is indicative for the presence of the Brachyspira sp. Sask30446 organism.

In an embodiment, detection of an est polynucleotide with at least 93.5% identity to SEQ ID NO: 27 or an est polypeptide with at least 95.5% identity to SEQ ID NO: 28 is indicative for the presence of the Brachyspira sp. Sask30446 organism.

In an embodiment, detection of a glpk polynucleotide with at least 95.5% sequence identity to SEQ ID NO: 29 or a glpk polypeptide with at least 99.5% sequence identity to SEQ ID NO: 30 is indicative for the presence of the Brachyspira sp. Sask30446 organism.

In an embodiment, detection of a pgm polynucleotide with at least 93.5% sequence identity to SEQ ID NO: 31 or a pgm polypeptide with at least 99.5% sequence identity to SEQ ID NO: 32 is indicative for the presence of the Brachyspira sp. Sask30446 organism.

In an embodiment, detection of a thi polynucleotide with at least 92.5% sequence identity to SEQ ID NO: 33 or a thi polypeptide with at least 95.5% sequence identity to SEQ ID NO: 34 is indicative for the presence of the Brachyspira sp. Sask30446 organism.

In an embodiment, detection of a 16S rRNA polnucleotide with at least 99.5% sequence identity to SEQ ID NO: 37 is indicative for the presence of the Brachyspira sp. Sask 30446 organism.

In a further embodiment, the level of Brachyspira sp. Sask30446 polynucleotide or polypeptide is quantified.

The methods can be employed to detect the presence or level of Brachyspira sp. Sask30446 in a subject that is healthy. For example, the methods can be used to screen healthy animals for the presence of low levels of Brachyspira sp. Sask30446. In an embodiment, the method is for screening subjects such as a herd of clinically healthy animals, for the presence of Brachyspira sp. Sask30446 for example to evaluate disease potential or status. For example a subclinically infected healthy pig could transmit Brachyspira sp. Sask30446 to other healthy animals in the farm or to another farm. As another example, a farm may test the feces of healthy animals to determine if the barn is contaminated/infected with Brachyspira sp. Sask30446. B. Sask30446 can for example be present in healthy animals at relatively low levels and may become problematic if its levels increase. In an embodiment, the method comprises identifying subjects with Brachyspira sp. Sask30446 and treating identified subjects prophylactically.

In an aspect, the disclosure includes a method of screening for, diagnosing or detecting colitis related to Brachyspira sp. Sask30446 and/or unrelated to B. hyodysenteriae and/or B. pilosicoli in a subject comprising determining a level of Brachyspira sp. Sask30446 polynucleotide or polypeptide in a sample of the subject, wherein detection of the Brachyspira sp. Sask30446 polynucleotide or polypeptide is indicative that the subject has colitis related to Brachyspira sp. Sask30446 and/or unrelated to B. hyodysenteriae and/or B. pilosicoli or has an increased risk of developing colitis related to Brachyspira sp. Sask30446 and/or unrelated to B. hyodysenteriae and/or B. pilosicoli.

In an embodiment, the determining step comprises determining a level of Brachyspira sp. Sask30446 NADPH oxidase, cpn60, est, plgk, pgm thi or 16S rRNA polynucleotide.

In an embodiment, the method comprises:

a) determining a level of one or more of:
i) a NADPH oxidase polynucleotide with at least 92.3% sequence identity with any one of SEQ ID NOs: 7-9 or a NADPH oxidase polypeptide with at least 92.5% sequence identity with SEQ ID NOs: 11 or 12;
ii) a cpn60 polynucleotide with at least 97.5% identity to SEQ ID NO: 25 or a cpn60 polypeptide with at least 98.5% identity to SEQ ID NO: 26;
iii) an est polynucleotide with at least 93.5% identity to SEQ ID NO: 27 or an est polypeptide with at least 95.5% identity to SEQ ID NO: 28
iv) a glpk polynucleotide with at least 95.5% sequence identity to SEQ ID NO: 29 or a glpk polypeptide with at least 99.5% sequence identity to SEQ ID NO: 30
v) a pgm polynucleotide with at least 93.5% sequence identity to SEQ ID NO: 31 or a pgm polypeptide with at least 99.5% sequence identity to SEQ ID NO: 32;
vi) a thi polynucleotide with at least 92.5% sequence identity to SEQ ID NO: 33 or a thi polypeptide with at least 95.5% sequence identity to SEQ ID NO: 34; and
vii) a 16S rRNA polnucleotide with at least 99.5% sequence identity to SEQ ID NO: 37;
   wherein detection of the polynucleotide or the polypeptide in a) is indicative the subject has colitis related to Brachyspira sp. Sask30446 and/or unrelated to B. hyodysenteriae and/or B. pilosicoli or an increased risk of developing colitis related to Brachyspira sp. Sask30446 and/or unrelated to B. hyodysenteriae and/or B. pilosicoli.

Brachyspira sp. Sask30446 was identified in high concentrations on direct examination of colon of the affected but not of the non-affected pigs. The levels of Brachyspira sp. Sask30446 in affected animals were quantifiable but were not quantifiable in healthy animals. For example, the limit of quantifiable is for example about 10⁴, optionally about 10³ copies of DNA/gram of tissue. Levels in non-affected pigs were negative or very low (e.g. unquantifiable). For example, high quantifiable levels (typically above 10⁶ or above target DNA copies per gram of tissue) of the novel Brachyspira species Sask30446 were detected by real-time PCR in the intestinal contents, colonic and/or caecal tissue of all 5 affected pigs, but B. sp. Sask30446 was either not detectable or was present at levels that were too low to be quantifiable in the intestinal tissues or contents of all 4 non-affected pigs. In another embodiment, the level associated with disease is at least 10⁵ or at least 10⁶ DNA copies/gram of tissue.

Accordingly, in an embodiment, the sample comprises colon cells and/or colon contents.

In an embodiment, the colitis is severe mucohaemorrhagic colitis related to Brachyspira sp. Sask30446 and/or unrelated to B. hyodysenteriae and B. pilosicoli.

Accordingly, in an embodiment, the disclosure includes a method of diagnosing severe mucohaemorrhagic colitis related to Brachyspira sp. Sask30446 and/or unrelated to B. hyodysenteriae and B. pilosicoli in a subject comprising:

a) determining a level of one or more in a sample from the subject:
i) a NADPH oxidase polynucleotide with at least 92.3% sequence identity with SEQ ID NOs: 7-9 or a NADPH oxidase polypeptide with at least 92.5% sequence identity with SEQ ID NOs: 11 or;
ii) a cpn60 polynucleotide with at least 97.5% identity to SEQ ID NO: 25 or a cpn60 polypeptide with at least 98.5% identity to SEQ ID NO: 26;
iii) an est polynucleotide with at least 93.5% identity to SEQ ID NO: 27 or an est polypeptide with at least 95.5% identity to SEQ ID NO: 28
iv) a glpk polynucleotide with at least 95.5% sequence identity to SEQ ID NO: 29 or a glpk polypeptide with at least 99.5% sequence identity to SEQ ID NO: 30
v) a pgm polynucleotide with at least 93.5% sequence identity to SEQ ID NO: 31 or a pgm polypeptide with at least 99.5% sequence identity to SEQ ID NO: 32;
vi) a thi polynucleotide with at least 92.5% sequence identity to SEQ ID NO: 33 or a thi polypeptide with at least 95.5% sequence identity to SEQ ID NO: 34; and
vii) a 16S rRNA polnucleotide with at least 99.5% sequence identity to SEQ ID NO: 37;
   wherein detection of the polynucleotide or the polypeptide in a) is indicative the subject has severe mucohaemorrhagic colitis related to Brachyspira sp. Sask30446 and/or unrelated to B. hyodysenteriae and B. pilosicoli or is at an increased risk of developing severe mucohaemorrhagic colitis related to Brachyspira sp. Sask30446 and/or unrelated to B. hyodysenteriae and B. pilosicoli.

In an embodiment, the subject is a pig.

It is also demonstrated herein that affected animals have an increase in the level of the Brachyspira species associated with colitis (e.g. Brachyspira sp. Sask30446) relative to an unaffected subject (e.g. unaffected by colitis) suggesting an increased level is associated with disease. For example, using sensitive techniques very low levels of Brachyspira sp. Sask30446 NADPH oxidase levels may be detected in intestinal tissue and/or intestinal contents of unaffected animals, however, affected animals have an increased level of Brachyspira sp. Sask30446 NADPH oxidase that is about 1 to 3 logs increased (e.g. the target copy number per gram of tissue or contents is increased up to 1 to 3 logs or about 10 to 1000 fold, in affected compared to control unaffected animals). Accordingly, another embodiment includes a method for screening for, diagnosing or detecting colitis related to Brachyspira sp. Sask30446 and/or unrelated to B. hyodysenteriae and/or B. pilosicoli in a subject comprising:
a. quantitating a level of a Brachyspira sp. Sask 30446 polynucleotide and/or polypeptide in a sample of the subject,
b. comparing the level of the Brachyspira sp. Sask30446 polynucleotide or polypeptide to a control,
wherein detection of an increased level of the Brachyspira sp. Sask30446 polynucleotide and/or polypeptide compared to the control, is indicative the subject has colitis related to Brachyspira sp. Sask30446 and/or unrelated to B. hyodysenteriae and/or B. pilosicoli.

In an embodiment, the Brachyspira sp. Sask30446 polynucleotide or polypeptide is selected from NADPH oxidase, cpn60, est, plgk, pgm, thi and 16S rRNA. In an embodiment, the NADPH oxidase polynucleotide has at least 92.5% sequence identity with SEQ ID NOs: 7-9 or a NADPH polypeptide has at least 92.5% sequence identity with SEQ ID NOs: 11 or 12, the cpn60 polynucleotide has at least 97.5% identity to SEQ ID NO: 25 or the cpn60 polypeptide has at least 98.5% identity to SEQ ID NO: 26; the est polynucleotide has at least 93.5% identity to SEQ ID NO: 27 or the est polypeptide has at least 95.5% identity to SEQ ID NO: 28; the glpk polynucleotide has at least 95.5% sequence identity to SEQ ID NO: 29 or the glpk polypeptide has at least 99.5% sequence identity to SEQ ID NO: 30; the pgm polynucleotide has at least 93.5% sequence identity to SEQ ID NO: 31 or the pgm polypeptide has at least 99.5% sequence identity to SEQ ID NO: 32; the thi polynucleotide has at least 92.5% sequence identity to SEQ ID NO: 33 or the thi polypeptide has at least 95.5% sequence identity to SEQ ID NO: 34; and/or the 16S rRNA polnucleotide has at least 99.5% sequence identity to SEQ ID NO: 37.

In an embodiment, the sequence identity of the Brachyspira sp. Sask30446 polynucleotide or polypeptide is selected from at least 92.5%, 93.5%, 94.5%, 95.5%, 96.5%, 97.5%, 98.5%, 99%, 99.5% or 100% identical to a Brachyspira sp. Sask30446 polynucleotide disclosed herein.

In an embodiment, the level of polynucleotide or polypeptide is significantly increased.

In an embodiment, the level of the Brachyspira sp. Sask30446 polynucleotide or polypeptide (e.g. target copies per gram of tissue or contents) is increased at least 2 fold, at least 3 fold, at least 4 fold, at least 5 fold, at least 6 fold, at least 7 fold, at least 8 fold, at least 9 fold or at least 10 fold. In an embodiment, the level of the Brachyspira sp. Sask30446 polynucleotide or polypeptide is increased at least 20 fold, at least 50 fold, at least 100 fold, at least 500 fold, at least 1000 fold, at least 2500 fold, at least 5000 fold or at least 10 000 fold. In an embodiment, the level of the Brachyspira sp. Sask30446 polynucleotide or polypeptide is increased at least 1 log, at least 2 log, at least 3 log or at least 4 log.

For example, it is also demonstrated that at least 10⁵ copies of Sask30446 NADPH oxidase polynucleotide per gram of sample are quantifiable in subject samples affected with the colitis related to Brachyspira sp. Sask30446 and/or unrelated to B. hyodysenteriae and/or B. pilosicoli. Accordingly, an embodiment includes a method for screening for, diagnosing or detecting colitis related to Brachyspira sp. Sask30446 and/or unrelated to B. hyodysenteriae and/or B. pilosicoli in a subject wherein the level of the Brachyspira sp. Sask30446 polynucleotide or polypeptide polynucleotide is quantitated per gram of a sample from the subject.

In an embodiment, the control is a predetermined or cut-off level. For example, samples comprising a level above the cut-off level are identified as associated with colitis related to Brachyspira sp. Sask30446 and/or unrelated to B. hyodysenteriae and/or B. pilosicoli. In an embodiment, the cut-off level is about 10³, about 10⁴, about 5×10⁴, about 10⁵, about 2×10⁵, about 5×10⁵, about 1×10⁶, about 2×10⁶, or about 5×10⁶ copies per gram. A number of techniques are known in the art for determining the presence or levels of a polynucleotide. For example, the polynucleotide can by amplified for example by PCR, detecting by hyrbridization for example by Southern analysis or in situ hybridization. Other techniques include, quantitative real time PCR, multiplex ligation dependent probe amplification (MLPA), nucleic acid sequence based amplification (NASBA) and/or real time NASBA. As used herein “NASBA” refers to a sensitive isothermal transcription-based amplification method used for example for RNA research. NASBA technology is optionally applied to single nucleotide polymorphism (SNP) analysis using human genomic DNA as a template. For example combination of DNA NASBA with multiplex hybridization of specific molecular beacons makes it possible to discriminate the presence of polynucleotides of interest.

In an embodiment, the method comprises in situ hybridization.

In an embodiment, the method comprises quantitative PCR, such as real time PCR. In another embodiment, the PCR method comprises SYBR green real time PCR. SYBR green is a fluorescent dye and intercalating agent that binds double-stranded DNA. During SYBR green real-time PCR, SYBR green dye is added to the PCR reaction and the thermocycler used is equipped with a camera that permits real-time monitoring of fluorescence accumulation during the run (each well or tube is measured at each cycle of the PCR). Real-time PCR assays can for example be more sensitive than conventional, end-point, PCR (e.g. where the products are analysed on an agarose gel after the reaction is completed).

In an embodiment, the NADPH oxidase polynucleotide is amplified in an amplification reaction such as PCR, RT-PCR, optionally qPCR or qRT-PCR, employing an oligonucleotide primer or primer pair that hybridizes to the NADPH oxidase polynucleotide or complement thereof.

In an embodiment, the method comprises use of a species-specific primer or primer pair for exampleprimer pair SEQ ID NOs: 3 and 4 or SEQ ID NOs: 5 and 6. A person skilled in the art would readily be able to identify and test species-specific primers for the Brachyspira sp. Sask30446 sequences provided herein. For example a person skilled in the art having the Brachyspira sp. Sask30446 could identify stretches of contiguous sequence that comprise one or more differing nucleotides compared to known Brachyspira sp. species to design primers. The primers could be tested to ensure species-specificity using methods known in the art.

In an embodiment, the method comprises amplifying at least 20 contiguous nucleotides of a NADPDH oxidase polynucleotide, the amplified polynucleotide having greater than 92.5% sequence identity to any one of SEQ ID NOs: 7 to 9.

In an embodiment, the primer comprises any one of SEQ ID NOs:3 to 6. In an embodiment, the primer pair is selected from SEQ ID NOs: 3 and 4 or SEQ ID NOs: 5 and 6.

In an embodiment, the method comprises amplifying at least 20 contiguous nucleotides of a cpn60 polynucleotide, the amplified polynucleotide has at least 97.5% identity to SEQ ID NO: 25 or the translation product of the polynucleotide has at least 98.5% identity to SEQ ID NO: 26.

In an embodiment, the method comprises amplifying at least 20 contiguous nucleotides of an est polynucleotide, the amplified polynucleotide has at least 93.5% identity to SEQ ID NO: 27 or the translation product of the polynucleotide has at least 95.5% identity to SEQ ID NO: 28.

In an embodiment, the method comprises amplifying at least 20 contiguous nucleotides of a glpk polynucleotide, the amplified polynucleotide has at least 95.5% identity to SEQ ID NO: 29 or the translation product of the polynucleotide has at least 99.5% identity to SEQ ID NO: 30.

In an embodiment, the method comprises amplifying at least 20 contiguous nucleotides of a pgm polynucleotide, the amplified polynucleotide has at least 93.5% identity to SEQ ID NO: 31 or the translation product of the polynucleotide has at least 99.5% identity to SEQ ID NO: 32.

In an embodiment, the method comprises amplifying at least 20 contiguous nucleotides of a thi polynucleotide, the amplified polynucleotide has at least 92.5% identity to SEQ ID NO: 33 or the translation product of the polynucleotide has at least 95.5% identity to SEQ ID NO: 34.

In an embodiment, the method comprises amplifying at least 20 contiguous nucleotides of a 16S rRNA polynucleotide, the amplified polynucleotide has at least 99.5% identity to SEQ ID NO: 37 or its translation product of the amplified polynucleotide has at least 99.5% identity to the translation product of SEQ ID NO: 37.

In an embodiment, the cpn60, est, glpk, pgm, thi, 16S rRNA polynucleotide is amplified with a primer or primer pair that are species-specific.

The novel Brachyspira species can also be detected by using primers that amplify Brachyspira polynucleotides in a number of species and probing the amplified sequence with a Brachyspira sp. Sask30446 polynucleotide specific probe. Alternatively the amplified polynucleotide can be sequenced and the sequence compared (or translated and compared when comparing to amino acid reference sequences) to a reference sequence for example any one of the Brachyspira sp. Sask30446 sequences provided here.

Accordingly, in an embodiment the method comprises:
a) amplifying at least 20 contiguous nucleotides of a Brachyspira sp. Sask30446 polynucleotide, using an oligonucleotide primer or primer set;
b) hybridizing the amplified sequence with a polynucleotide probe which specifically binds the Brachyspira sp. Sask30446 polynucleotide.

In another embodiment, the method comprises:
a) amplifying at least 20 contiguous nucleotides of a Brachyspira sp. Sask30446 polynucleotide, using an oligonucleotide primer or primer set;
b) sequencing the amplified polynucleotide; and
c) comparing the sequence to a reference sequence.
   wherein a sequence with at least 92.5%, 93.5%, 94.5%, 95.5%, 96.5%, 97.5%, 98.5%, 99% or 99.5% sequence identity to at the least 20 contiguous nucleotides of the reference sequence is indicative of the presence of the Brachyspira species unrelated to B. hyodysenteriae and/or B. pilosicoli (e.g. is indicative of the presence of Brachyspira sp. Sask30446).

In an embodiment, the method comprises use of genus, multispecies or universal primers SEQ ID NOs: 1 and 2; SEQ ID NOs: 15 and 16; SEQ ID NO ID NOs: 17 and 18; SEQ ID NOs: 19 and 20; SEQ ID NOs: 21 and 22; and/or SEQ ID NOs: 35 and 36, for example as demonstrated in the Examples. As mentioned, in embodiments where multispecies or universal primers are employed, the resulting PCR products can be sequenced and compared to one or more reference sequences (e.g. where the species information is known). For example, in methods employing use of primers 15 and 16 to amplify est, polynucleotide, the amplified product is sequenced and compared to a Brachyspira sp. Sask30446 est sequence such as in SEQ ID NOs: 27 or 28 (e.g. the polynucleotide is translated into amino acid sequence using an appropriate reading frame).

In an embodiment, the method comprises amplifying at least 20 contiguous nucleotides of a NADPH polynucleotide, the amplified polynucleotide has at least 92.5% identity to any one of SEQ ID NOs: 7-9 or the translation product has at least 92.5% identity to SEQ ID NO: 11 or 12.

In an embodiment, a primer or primer set for amplifying NADPH comprises SEQ ID NOs: 1 and/or 2. In an embodiment, the oligonucleotide primer set is selected from: a first oligonucleotide comprising a sequence of SEQ ID NO: 1 and a second oligonucleotide comprising a sequence of SEQ ID NO: 2; a first oligonucleotide comprising a sequence of SEQ ID NO: 3 and a second oligonucleotide comprising a sequence of SEQ ID NO:4; and a first oligonucleotide comprising a sequence of SEQ ID NO: 5 and a second oligonucleotide comprising a sequence of SEQ ID NO:6.

In an embodiment, the method comprises amplifying at least 20 contiguous nucleotides of a cpn60 polynucleotide, the amplified polynucleotide has at least 97.5% identity to SEQ ID NO: 25 or the translation product has at least 98.5% identity to SEQ ID NO: 26.

In an embodiment, the primer set for amplifying cpn60 comprises SEQ ID NOs: 23 and 24. In an embodiment, the primer pair comprises a species-specific primer pair.

In an embodiment, the method comprises amplifying at least 20 contiguous nucleotides of an est polynucleotide, the amplified polynucleotide has at least 93.5% identity to SEQ ID NO: 27 or the translation product has at least 95.5% identity to SEQ ID NO: 28.

In an embodiment, the primer pair comprises SEQ ID NO:15 and 16. In an embodiment, the primer pair comprises a species-specific primer pair.

In an embodiment, the method comprises amplifying at least 20 contiguous nucleotides of a glpk polynucleotide, the amplified polynucleotide has at least 95.5% identity to SEQ ID NO: 29 or the translation product has at least 99.5% identity to SEQ ID NO: 30.

In an embodiment, the primer pair comprises SEQ ID NO:17 and 18. In an embodiment, the primer pair comprises a species-specific primer pair.

In an embodiment, the method comprises amplifying at least 20 contiguous nucleotides of a pgm polynucleotide, the amplified polynucleotide has at least 93.5% identity to SEQ ID NO: 31 or the translation product has at least 99.5% identity to SEQ ID NO: 32.

In an embodiment, the primer pair comprises SEQ ID NO:19 and 20. In an embodiment, the primer pair comprises a species-specific primer pair.

In an embodiment, the method comprises amplifying at least 20 contiguous nucleotides of a thi polynucleotide, the amplified polynucleotide has at least 92.5% identity to SEQ ID NO: 33 or the translation product has at least 95.5% identity to SEQ ID NO: 34.

In an embodiment, the primer pair comprises SEQ ID NO:21 and 22. In an embodiment, the primer pair comprises a species-specific primer pair.

In an embodiment, the method comprises amplifying at least 20 contiguous nucleotides of a 16S rRNA polynucleotide, the amplified polynucleotide has at least 99.5% identity to SEQ ID NO: 37 or its translation product.

In an embodiment, the primer pair comprises SEQ ID NOs: 35 and 36. In an embodiment, the primer pair comprises a species-specific primer pair

In an embodiment, at least 40, at least 60, at least 80, at least 100, at least 120, at least 140, at least 160, at least 180 or at least 200 contiguous nucleotides are amplified. In an embodiment, 200-250, 250-300, 300-350, 350-400 or more contiguous nucleotides are amplified. The number of contiguous nucleotides amplified can be any number that is practical for example, practical for detection and/or quantification.

In another embodiment, the method comprises contacting the sample with an analyte specific binding agent (ASBA) which specifically binds a Brachyspira sp. Sask30446 polynucleotide or polypeptide; and determining the level of the polynucleotide or polypeptide in the sample.

The ASBA, when a polynucleotide is characterized in that it binds to a Brachyspira sp. Sask30446 polynucleotide such as a NAPDH oxidase polynucleotide, under stringent conditions, for example as described herein.

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

In an embodiment, the ASBA is selected from a NADPH oxidase ASBA, a cpn60 ASBA, an est ASBA, a glpk ASBA, a pgm ASBA a thi ASBA and a 16S ASBA.

In an embodiment, the NADPH oxidase analyte specific binding agent is a polynucleotide probe comprising:
a) at least 20 contiguous nucleotides of any one of SEQ ID NOs:7 to 9;
b) a sequence with at least 92.5% identity to 20 contiguous nucleotides of any one of SEQ ID NOs: 7 to 9; or
c) a complementary sequence of a) or b).

In an embodiment, the cpn60 analyte specific binding agent is a polynucleotide probe comprising:
a) at least 20 contiguous nucleotides of SEQ ID NO:25;
b) a sequence with at least 97.5% identity to 20 contiguous nucleotides of SEQ ID NO: 25; or
c) a complementary sequence of a) or b).

In an embodiment, the est analyte specific binding agent is a polynucleotide probe comprising:
a) at least 20 contiguous nucleotides of SEQ ID NO:27;
b) a sequence with at least 93.5% identity to 20 contiguous nucleotides of SEQ ID NO: 27; or
c) a complementary sequence of a) or b).

In an embodiment, the glpk analyte specific binding agent is a polynucleotide probe comprising:
a) at least 20 contiguous nucleotides of SEQ ID NO:29;
b) a sequence with at least 95.5% identity to 20 contiguous nucleotides of SEQ ID NO: 29; or
c) a complementary sequence of a) or b).

In an embodiment, the pgm analyte specific binding agent is a polynucleotide probe comprising:
a) at least 20 contiguous nucleotides of SEQ ID NO:31;
b) a sequence with at least 93.5% identity to 20 contiguous nucleotides of SEQ ID NO: 31; or
c) a complementary sequence of a) or b).

In an embodiment, the thi analyte specific binding agent is a polynucleotide probe comprising:
a) at least 20 contiguous nucleotides of SEQ ID NO:33;
b) a sequence with at least 92.5% identity to 20 contiguous nucleotides of SEQ ID NO: 33; or
c) a complementary sequence of a) or b).

In an embodiment, the 16S rRNA analyte specific binding agent is a polynucleotide probe comprising:
a) at least 20 contiguous nucleotides of SEQ ID NO:37;
b) a sequence with at least 99.5% identity to 20 contiguous nucleotides of SEQ ID NO: 37; or
c) a complementary sequence of a) or b).

In an embodiment, the polynucleotide probe comprises at least 30, at least 40, at least 50, at least 60, at least 70, at least 80, at least 90, at least 100 or more nucleotides of any one of SEQ ID NOs: 7 to 9, 25, 27, 29, 31, 33 or 37.

For example a variety of techniques are known in the art for detecting a polynucleotide sequence, including for example microarrays, Restriction Fragment Length Polymorphism, Southern Blots, SSCP, dHPLC, single nucleotide primer extension, allele-specific hybridization, allele-specific primer extension, oligonucleotide ligation assay, and invasive signal amplification, Matrix-assisted laser desorption/ionization time-of-flight (MALDI-TOF) mass spectrometry, polymerase chain reaction, loop-mediated isothermal amplification (LAMP) and Fluorescence polarization (FP). Such methods optionally employ the isolated nucleic acid compositions of the disclosure.

In an embodiment, the method comprises:
a) contacting the sample with the polynucleotide probe; and
b) detecting the level of the Brachyspira sp. Sask304466 polynucleotide, the Brachyspira sp. Sask30446 polynucleotide comprising a sequence with at least 92.5% identity to any one of SEQ ID NOs: 7 to 9, 25, 27, 29, 31, 33 or 37.

In another embodiment, the analyte specific binding agent is an antibody specific for a polypeptide comprising a sequence of SEQ ID NOs: 11 12, 26, 28, 30, 32 and 34 or encoded by any one of SEQ ID NOs: 7 to 9, 25, 27, 29, 31, 33 and 37.

In an embodiment, the method comprises:
a) contacting the sample with the antibody; and
b) detecting the level of the Brachyspira sp. Sask30446 polypeptide, the NADPH polypeptide comprising a sequence with at least 92.5% identity with SEQ ID NOs:11, 12, 26, 28, 30, 32 and 34 or a sequence encoded by any one of SEQ ID NOs: 7 to 9, 25, 27, 29, 31, 33 and 37.

In an embodiment the isolated polypeptides, antibodies or antibody fragments are labeled with a detectable marker.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a secondary antibody that is specific for the isolated protein described in the application and contains a detectable label is useful to detect the isolated polypeptide described in the application.

A person skilled in the art would be familiar with a number of antibody based methods for detecting a polypeptide or level thereof, including for example, Western blot, ELISA and/or immunohistochemistry. In an embodiment, the method comprises using Western blot, immunoprecipitation, ELISA and immunohistochemistry and/or immunocytochemistry.

In an embodiment, the method comprised obtaining a sample from the subject. The sample is optionally processed e.g. sample lysed and/or DNA is isolated and is used to determine a level of a Brachyspira sp. Sask30446 polynucleotide or polypeptide.

In an embodiment, the sample comprises colon cells, fecal material and/or gastrointestinal content. In an embodiment, the sample comprises contents, cells, and/or tissues derived from stomach, duodenum, ileum, jejunum, colon, caecum and/or rectum. In an embodiment, the sample is a fixed sample, for example a fixed tissue block or tissue section adhered to a slide. The sample can be fixed as in tissue, fresh or frozen as in contents or tissue, or preserved in other reagents and compounds, for example glutaraldehyde for electron microscopy evaluation of tissue. In an embodiment, the sample is from a subject with colitis. In an embodiment, the sample is from a healthy animal.

For example, for detecting Brachyspira sp. Sask30446 or diagnosing a Brachyspira sp. Sask30446 infection in a live animal, a fecal sample is obtained. For post-mortem diagnosis, colon tissue or colon contents can be assayed. In an embodiment, the species-specific nox-based primer set in either conventional or quantitative real-time PCR can be applied with SYBR green and the presence of the DNA is quantitated. Other gene targets can also be used. For example, PCR can used with the broad-spectrum primers to amplify any other Brachyspira sp. Sask30446 gene sequences, purifying any product produced (if of the appropriate size), determining its DNA sequence and comparing that sequence to a reference sequence database for identification. Alternatively, species-specific primers could be easily generated for the other genes (est, glpk, pgm, thi) similar to as developed for Bnox.

Confirmation of identity of cultured isolates can be accomplished for example, through PCR with JH0224/JH0225 (including appropriate positive and negative controls) or PCR from cultured isolates with universal primers or Brachyspira MLST primers followed by DNA sequencing and comparison of the resulting sequence(s) to a reference database comprising reference sequences.

In an embodiment, the molecular diagnostic results are considered in the context of the clinical information.

In an embodiment, the sample is cultured, for example to obtain a colony, prior to determining a level of a Brachyspira sp. Sask30446 polynucleotide or polypeptide.

In an embodiment, the sample is cultured according to a method described herein.

A further aspect provides a method of growing a Brachyspira sp. Sask30446 organism comprising inoculating a blood agar medium (BAM) and incubating at a temperature between 25-44° C.

In an embodiment, the temperature is between about 35-44° C. (e.g. 35° C., 36° C., 37° C., 38° C., 39° C., 40° C., 41° C., 42° C., 43° C., 44° C., or any 0.1° C. increment between 35° C. and 44.9° C.), between about 38-44° C., between 40-43° C. or between 39-42 C. In another embodiment, the temperature is about 35° C., about 36° C., about 37° C. or about 38 C. In an embodiment, the temperature is about 39° C., about 40° C., about 41° C., about 42° C., about 43° C. or about 44° C. In an embodiment, the temperature is about 42° C.

The BAM comprises blood. In an embodiment, the blood is mammalian blood. In an embodiment, the blood is horse blood. In another embodiment the blood is sheep blood. In a further embodiment, the blood is defibrinated blood. In an embodiment, the blood is whole blood.

In an embodiment, the blood agar medium comprises 5-10% mammalian blood. In an embodiment, the BAM comprises about 5%, about 6%, about 7%, about 8%, about 9% or about 10% blood.

In an embodiment, the BAM comprises blood agar base and mammalian blood. In an embodiment, the blood agar base is blood agar base no. 2 (Oxoid). For example, the composition of blood agar base No. 2 comprises 15 g/L proteose peptone, 2.5 g/L liver digest, 5.0 g/L yeast extract, 5.0 g/L sodium chloride, 12 g/L agar. These are all generic products that could be purchased from any chemical/media supplier for example Difco or Becton Dickinson (BD). Each of these components can be varied, for example by about 50%, about 40%, about 30%, about 20% or about 10%.

In an embodiment, the blood agar base is 20 to 60 g/L for example 30, 35, 40, 45 or 50 g/L of the BAM. In an embodiment, the BAM comprise beef extract. In an embodiment, the beef extract is about 1.5 g/L, about 2 g/L about 2.5 g/L, about 3 g/L about 3.5 g/L, about 4 g/l or about 4.5 g/L. In a further embodiment the BAM comprises Bacto Peptone (Difco) optionally at about 2.5 g/l, about 3 g/L, about 3.5 g/L, about 4 g/L, about 4.5 g/L, about 5 g/L, about 5.5. g/L, about 6 g/L, about 6.5 g/L about 7 g/L or about 7.5 g/L. In an embodiment, the BAM comprises antibiotics such as spectomycin and/or rifampin.

In an embodiment, the BAM comprises Blood Agar Base no. 2 (Oxoid) (40 g l⁻¹), Beef Extract (Difco) (3 g l⁻¹) and Bacto Peptone (Difco) (5 g l⁻¹)), supplemented with defibrinated horse blood (7%), spectinomycin (400 mg ml⁻¹) and rifampin (15-30 mg ml⁻¹) (e.g. BAM-SR) (Calderaro et al., 2005).

In an embodiment, the BAM is comprised in a plate, eg. a blood agar plate.

In an embodiment, the agar is inoculated directly for example with a sample of an infected subject, for example a sample of colon tissue from an infected pig incubated in anaerobic media is used to inoculate the agar culture. In another embodiment, the inoculation comprises placing a filter comprising sample on BAM, for example filtering the sample (e.g. colon tissue in anaerobic medium) through a 0.45 μm filter such that the sample is retained on the filter and placing the filter on the BAM. For example, the tissue is placed on top of a filter so that the organisms have to swim through the filter to reach the agar medium. Non-motile organisms and other debris are retained on the filter. In another embodiment, the method comprises inoculation of media with broth, e.g. anaerobic broth, that was incubated with the tissue sample, for example at room temperature for 30 minutes, through a filter. For example, 2 drops of broth are dripped onto filter placed on agar media and incubated.

In an embodiment, the BAM is incubated for at least 2 days, at least 4 days, at least 6 days, at least 8 days or at least 10 days. For example, after 8 days of anaerobic incubation, Brachyspira sp. Sask30446 is visible as a weakly hemolytic (on horse blood), tiny, clear, wet/glistening, “fried egg” colony. The colonies are less than 1 mm in diameter (FIG. 5).

In an embodiment, the cultured Brachyspira sp. Sask30446 is Gram stained. Gram stains of isolates from agar plates show pleiomorphic, Gram negative spirochetes with tapered ends. Length of the cells is variable, 2-20 μm (FIG. 6). Spriochetes with the same morphology are abundant in samples of colon contents (feces) from pigs with hemorrhagic colitis that also test positive by the Sask30446 qPCR assay (primers JH0224/JH0225) with counts of ˜1×10⁷ Sask30446 organisms per gram of feces or colon contents (FIG. 7).

The culture characteristics can be used in determining the presence of Brachyspira sp. Sask30446. In an embodiment, the method of screening for or detecting a presence of Brachyspira sp. Sask30446 organism in a sample from a subject comprises:
a) obtaining a sample from the subject;
b) inoculating a blood agar medium (BAM) with the sample and incubating at a temperature of 25-44 C;
   wherein the presence of weakly hemolytic (e.g. on horse blood), tiny, clear, wet/glistening, “fried egg” colonies is indicative for the presence of Brachyspira sp. Sask30446 in the subject.

The culture characteristics can be used for example to determine the presence of Brachyspira sp. Sask30446.

In an embodiment, the method of screening for or detecting a presence of Brachyspira sp. Sask30446 organism in a sample from a subject comprises:

a) obtaining a sample from the subject;

b) inoculating a blood agar medium (BAM) with the sample and incubating at a temperature of 25-44 C;

c) determining a level of Brachyspira sp. Sask30446 polynucleotide or polypeptide in any cell or colony obtained in step b)

wherein the presence of weakly hemolytic (e.g. on horse blood), tiny, clear, wet/glistening, “fried egg” colonies and/or the presence of a Brachyspira sp. Sask30446 polynucleotide or polypeptide is indicative for the presence of Brachyspira sp. Sask 30446 in the subject.

III. Compositions and Kits

Another aspect of the disclosure includes an isolated polynucleotide comprising at least 15 contiguous nucleotides of anyone of a) SEQ ID NOs: 3-9, 25, 37, 29, 31, 33, 35 and 37, the at least 15 contiguous nucleotides comprising any sequence of nucleotides with at least one difference from known Brachyspira sequences, b) a sequence at least 92.5% identical to any one of SEQ ID NOs: 3-9, 25, 37, 29, 31, 33, 35 and 37; or a complement of a) or b). In an embodiment, the isolated polynucleotide comprises at least 20, at least 30, at least 40, at least 50, at least 100, at least 150, at least 200, at least 300, at least 400, at least 500, at least 600, at least 700 or at least 800 nucleotides of any one of a) SEQ ID NOs: 3-9, 25, 37, 29, 31, 33, 35 and 37, b) a sequence at least 92.5% identical to any one of SEQ ID NOs: 3-9, 25, 37, 29, 31, 33, 35 and 37; or a complement of a) or b).

Another embodiment includes an isolated polypeptide comprising at least 10 contiguous amino acids of SEQ ID NOs: 11, 12, 26, 28, 30, 32 and 34 or encoded by any one of SEQ ID NOs: 7 to 9, 25, 37, 29, 31, 33, 35 and 37 or a sequence at least 92.5% identical to a polypeptide encoded by any one of SEQ ID NOs: 3-9, 25, 37, 29, 31, 33, 35 and 37. In an embodiment, the isolated polypeptide comprises anywhere between 10 and 270 amino acids. In an embodiment, the isolated polypeptide comprises at least 20, at least 30, at least 40, at least 50, at least 60, at least 70, at least 80, at least 90 or at least 100 amino acids. In another embodiment, the isolated polypeptide comprises as least 100, at least 150, at least 200, or at least 250 amino acids.

A person skilled in the art will readily recognize that the probes, primers, polynucleotide and polypeptides can by made using recombinant molecular biology techniques. For example, a probe can be cloned into a cloning vector and propagated in any number or organisms such as E. coli etc., the plasmid can be recovered and the probe isolated using restriction endonucleases.

A further aspect comprises an isolated antibody specific for the isolated polypeptide described herein. For example, the antibody is considered specific if it preferentially binds to a polypeptide encoded by any one of SEQ ID NOs: 7 to 9, 25, 37, 29, 31, 33, 35 and 37 compared to a polypeptide encoded by SEQ ID NO:10 (e.g. a known related species).

A further aspect includes a composition comprising a polynucleotide comprising at least 15 contiguous nucleotides of anyone of a) SEQ ID NOs: 3-9, 25, 37, 29, 31, 33, 35 and 37 b) a sequence at least 92.5% identical to any one of SEQ ID NOs: 3-9, 25, 37, 29, 31, 33, 35 and 37; or a complement of a) or b); a polypeptide comprising at least 10 contiguous amino acids of SEQ ID NOs: 11, 12, 26, 28, 30, 32 and 34 or encoded by any one of SEQ ID NOs: 7 to 9, 25, 37, 29, 31, 33, 35 and 37 or a sequence at least 92.5% identical to a polypeptide encoded by any one of SEQ ID NOs: 3-9, 25, 37, 29, 31, 33, 35 and 37 or an antibody specific for said polypeptide; and optionally a suitable diluent or carrier.

In an embodiment, the diluent or carrier comprises phosphate-buffered saline solution.

The composition can comprise at least 2 isolated polynucleotides, at least 2 polypeptides or at least 2 antibodies.

Also provided is a kit comprising an isolated polynucleotide comprising at least 15 contiguous nucleotides of anyone of a) SEQ ID NOs: 3-9, 25, 37, 29, 31, 33, 35 and 37, b) a sequence at least 92.5% identical to any one of SEQ ID NOs: 3-9, 25, 37, 29, 31, 33, 35 and 37; or a complement of a) or b); an isolated polypeptide comprising at least 10 contiguous amino acids of SEQ ID NOs: 11, 12, 26, 28, 30, 32 and 34 or encoded by any one of SEQ ID NOs: 7 to 9, 25, 37, 29, 31, 33, 35 and 37 or a sequence at least 92.5% identical to a polypeptide encoded by any one of SEQ ID NOs: 3-9, 25, 37, 29, 31, 33, 35 and 37 or an isolated antibody specific for said polypeptide; and one or more additional components selected for example from a polynucleotide useful as a positive or negative control; an analyte specific binding agent specific for a Brachyspira sp. polynucleotide or polypeptide; a standard, such as a molecular weight standard or positive control (e.g. plasmid or other vehicle comprising a Brachyspira sp. polynucleotide) and a reaction tube or plate. In an embodiment, the kit comprises a container, collection tube, diluent or buffer, for example for a saline buffer, a PCR buffer, nucleotides etc.

In an embodiment, the kit comprises an isolated polynucleotide or polypeptide with at least 93.5%, 94.5%, 95.5%, 96.5%, 97.5%, 98.5%, 99%, 99.5% or 100% sequence identity with any one of SEQ ID NOs: 1-37.

In an embodiment, the kit comprises primers JH0224/JH0225.

In another embodiment, the kit comprises a positive control such as a genomic DNA from Brachyspira sp. Sask30446 or a plasmid containing the target sequence.

In another embodiment, the kit comprises an internal positive PCR control either for confirmation of bacterial DNA in the sample (such as a universal primer set for amplifying 16S rRNA or cpn60) or for confirmation of Brachyspira spp. in the sample (e.g. primers JH0222/JH0223).

The kit can also for example comprise materials and solutions for extracting total genomic DNA, for example using a standard method.

In an embodiment, the kit comprises a multiplex kit comprising probes or primers for a panel of Brachyspira species, optionally for detecting multiple species including the novel species detected herein simultaneously. In an embodiment, the kit comprises species-specific PCR assays for B. hyodysenteriae, B. pilosicoli and/or B. innocens.

The following non-limiting examples are illustrative of the present disclosure:

### EXAMPLES

### Example 1

### Introduction

Five Brachyspira spp. commonly infect pigs 3 months of age and older. B. hyodysenteriae (Bh) and B. pilosicoli (Bp) cause swine dysentery and spirochetal colitis respectively, whereas B. innocens (Binn), and B. intermedia (Bint) are generally considered avirulent. B. murdochii (Bm) has only recently been associated with catarrhal colitis (1).

We have recently investigated several cases of severe mucohaemorrhagic typhlocolitis unrelated to Bh and Bp, and have identified a novel Brachyspira sp. (Brachyspira sp. Sask30446) in intestinal tissue and feces of affected pigs. A small case-control study has been performed in order to contrast the pathological and microbiological findings of affected and non-affected pigs. Described herein are case submissions from 2 unrelated farms experiencing a mucohaemorrhagic colitis associated with a novel Brachyspira species.

### Results

A number of cases of severe mucohaemorrhagic colitis and bloody diarrhea unrelated to B. hyodysenteriae and B. pilosicoli have been identified. Multiple submissions of pigs and feces were made from two such farms by the referring veterinarians from which we detected the presence of a novel Brachyspira sp. (as detailed below) in the intestinal content and feces of affected pigs. We have also completed detailed necropsy examinations on two affected and two non-affected pigs from one of these farms in which we have contrasted the clinical, pathological and microbiological findings. All pigs were in good body condition. The affected pigs demonstrated blood diarrhea, whereas the age-matched, non-affected demonstrated normal feces. The pathologic lesions in the 2 clinical cases were typical of swine dysentery: necrotizing haemorrhagic non-suppurative colitis+/−typhlitis. There were no remarkable lesions in the non-affected pigs. Bacterial cultures using Brachyspira-specific media and environmental conditions have been unsuccessful. The Brachyspira sp. was identified on direct examination of colon of the affected but not of the non-affected pigs. The novel Brachyspira species was detected by PCR in the intestinal contents and caecal tissue of all 4 pigs, but only in the colonic tissue of affected pigs. Testing for B. hyodysenteriae and B. pilosicoli was completed and shown to be negative for all pigs for all tissues. Other known intestinal swine pathogens including Lawsonia intracellularis, rotavirus, transmissible gastroenteritis and porcine circovirus were ruled out as was Porcine Reproductive and Respiratory Syndrome (PRRS) virus. Without wishing to be bound by theory, a hypothesis is that the novel Brachyspira may be a normal inhabitant of the pig's gut, but under some situations propagates and colonizes the colon resulting in a pathology and clinical disease.

### Farm H

Five submissions of fecal swabs, fixed tissues or carcasses were made from grow-finish pigs from a single source finisher barn exhibiting bloody diarrhea in 13 to 16 weeks old pigs. Pigs were vaccinated for PCV2 and erysipelas. Diets were medicated with 44 g/tonne (T) lincomycin from 10 to 16 weeks of age, and with 110 g/T lincomycin for 1 week if significant diarrhea was noted in a group. The total mortality rate attributable to bloody diarrhea was estimated to be 1% to 2%. Histologic examination confirmed a mucohaemorrhagic and suppurative colitis. No Salmonella sp. was isolated. PCR for Bh, Bp, Binn and Lawsonia intracellularis (Li) were negative. PCR for a conserved 939 bp fragment of the Brachyspira NADH oxidase 1 gene (Bnox1)(2) used to detect all Brachyspira sp. (Bsp) was positive (Table 1).

TABLE 1
Diagnostic results of Farm H pigs demonstrating bloody diarrhea and
colitis unrelated to B. hyodysenteriae and B. pilosicoli.

| | Type of | Bacterial culture | |
|---|---|---|---|
| Case | submission | (colon) | PCR results |
| 26620 | Fecal swabs | Strep sp., E. coli, | Bh, Binn, Bp: Neg |
| | | Acinetobacter sp. | Li: neg |
| 27021 | Fecal swabs | Neg | Bnox: Pos |
| 28403 | Fixed tissue | Strep sp, E. coli | Bnox: Neg |
| | Fecal swabs | | |
| 30446 | Carcasses | Mixed Bsp. | Bh & Bp: Neg, Bnox1: Pos |
| 12182 | Fecal swabs | Not performed | Bh, Binn, Bp: Neg |
| | | | Li: Neg |

Legend:
Bnox = Brachyspira NADH oxidase 1 gene;
Bh = B. hyodysenteriae,
Bi = B. innocens;
Bp = B. pilosicoli;
Bsp = Brachyspira sp.;
Li = Lawsonia intracellularis

### Farm D

Fresh and fixed tissues from 3 grow-finish pigs demonstrating bloody diarrhea were submitted from a single source nursery-finisher barn. The affected pigs had been vaccinated for PCV2 and were pulse medicated with 33 g/T tylosine (1 week of 3) from 9 to 15 weeks of age. The incidence of bloody diarrhea in 11 to 24 week old pigs was reported infrequent. Total annualized grow-finish mortality was estimated to be 3.5 to 4.5% on the farm. Histologic examination confirmed a catarrhal colitis. Bacterial cultures were negative for Bsp. and Salmonella sp. PCR for Bh, Bp and Binn were negative. Bnox PCR was positive in 1 case from which extracted tissue was retained (Table 2).

TABLE 2
Diagnostic results of Farm D pigs demonstrating bloody diarrhea and
colitis unrelated to B. hyodysenteriae and B. pilosicoli

| | | Bacterial culture | |
|---|---|---|---|
| Case | Type of submission | (colon) | PCR results |
| 17757 | Fresh & fixed tissue | E. coli, | Bh, Binn, Bp: Neg |
| | | Lactobacillus sp, | |
| | | Campylobacter sp | |
| 22958 | Fresh & fixed tissue | Neg | Bh, Binn, Bp: Neg |
| 28573 | Fresh & fixed tissue | E. coli | Bnox: Pos |

Legend:
Bnox = Brachyspira NADH oxidase 1 gene;
Bh = B. hyodysenteriae,
Bi = B. innocens;
Bp = B. pilosicoli

### Case-Control Submission (Farm D)

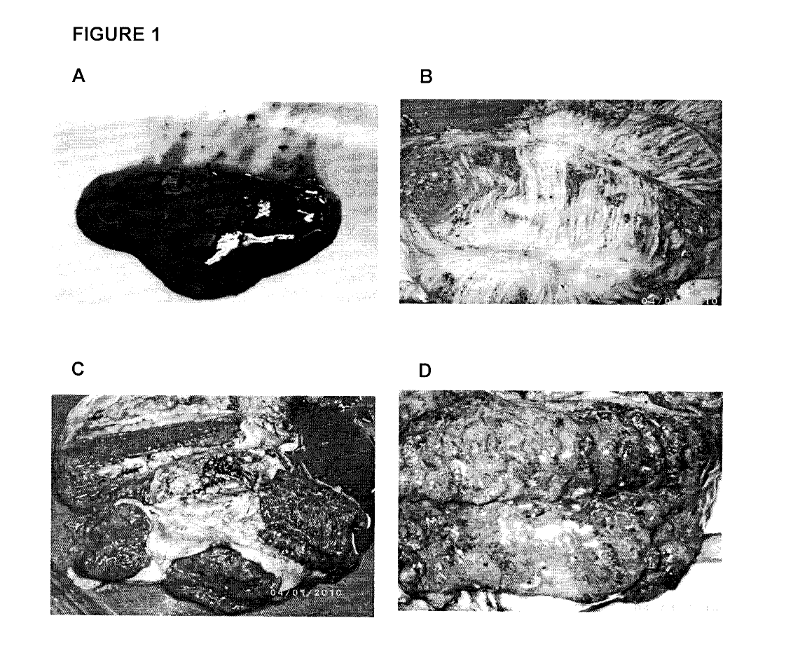

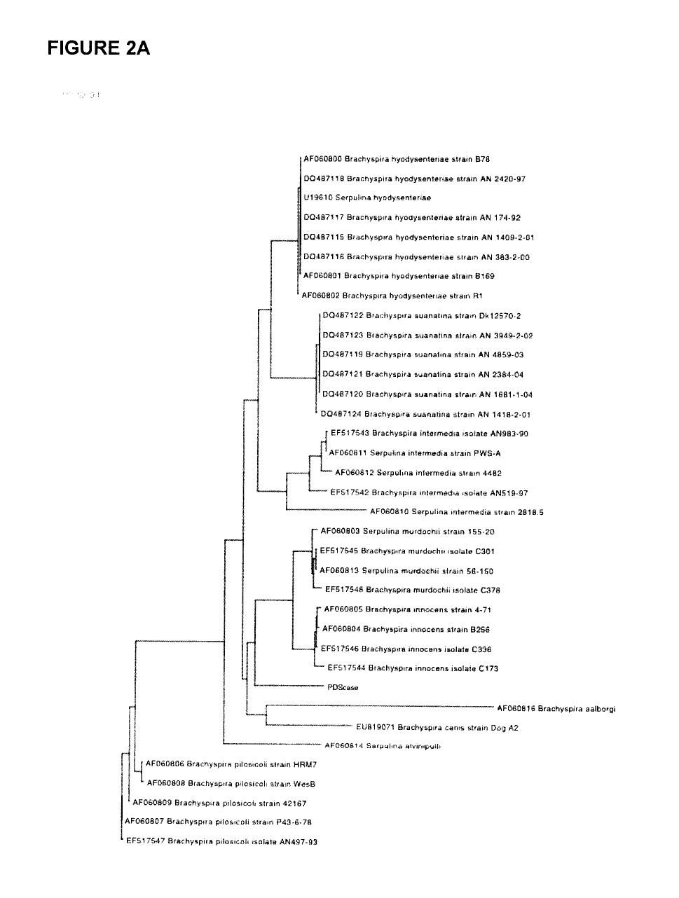

Four 50 kg pigs with (Case) or without (Ctrl) bloody diarrhea were examined. An acute mucohaemorrhagic typhlocolitis was found in Case pigs (FIG. 1). No enteritis or typhlocolitis was found in either Ctrl. Intestinal tissue from all was negative for Bh, Binn and Li by PCR, and negative for rotavirus and transmissible gastroenteritis by FAT. One unaffected pig (CTRL 30) had detectable Bp by PCR in colon and caecum. There is evidence in the literature of non-diarrheic pigs being positive for Bp (Heinonen M et al. 2000).

All were PRRS negative (IHC lung) and ¾ were PCV2 negative (IHC lymphoid tissue). No Salmonella sp. was cultured. Abundant (4+) Brachyspira-like organisms were identified by direct examination of colon of Case but not Ctrl pigs. A Brachyspira sp. Sask30446-specific PCR assay amplifying a 241 bp fragment of the Bnox gene was developed. Brachyspira sp. Sask30446 DNA was identified in colon and colonic contents of Case but not Ctrl pigs (Table 3). Brachyspira sp. Sask30446 DNA was also identified in the ileum of 1 Ctrl, and the Caecum of 1 Case.

TABLE 3
Brachyspria sp. Sask30446 PCR results on tissue and contents of
Case and Control (Ctrl) pigs

| | Jejunum | Ileum | Cecum | Colon |
|---|---|---|---|---|
| Pig | (tissue) | (tissue) | (tissue/contents) | (tissue/contents) |
| Ctrl 30 | − | + | −/− | −/− |
| Ctrl 33 | − | − | −/− | −/− |
| Case 31 | − | − | −/− | +/+ |
| Case 32 | − | − | +/− | +/+ |

### PCR and Sequence Analysis (Colon)

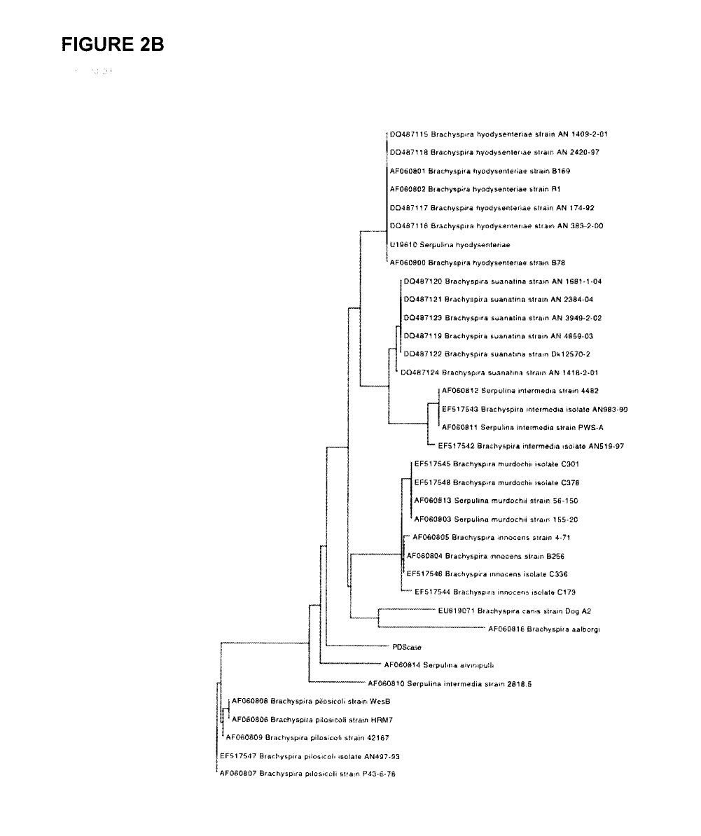

A species-specific PCR assay targeting B. hyodysenteriae was negative for all samples and a species-specific PCR assay targeting B. pilosicoli was negative except for 1 unaffected pig as mentioned above). PCR primers designed to amplify a region of the NADH oxidase 1 gene (nox1) of Brachyspira spp. (Rhode et al 2002) yielded a fragment of the expected size and direct sequencing of the nox1 amplicons yielded sequences with about 99% identity (for example SEQ ID NOs: 7-9. The nox1 sequence detected had a maximum of 92.3% identity to any previously characterized species. Phylogenetic analysis of relevant sequences (FIG. 2A) shows that the case sequence is distinct from other species which exhibit very little intraspecific diversity, and that its distance from other species is typical of interspecific distances for the genus Brachyspira (further discussed below). Based on the 810 bp alignment of Bnox sequences, PCR primers were designed (using SigOligo) to amplify a 241 bp fragment of the Brachyspira sp. Sask30446 nox1 gene.

(SEQ ID NO: 3)
5′-TCG CTA AAT TAT TCC AAC AAG GA-3′ [JH0224]
(SEQ ID NO: 4)
5′-AAA CGC ATT TCT ATT CCA GCA-3′ [JH0225]

Four pigs from an affected barn were brought to WCVM for necropsy and full diagnostic work-up. We received samples of GI tract (jejunum, ileum, colon and contents) from all pigs. Total DNA extraction performed on all tissues and some contents for PCR. Samples were blinded to PCR operator. Results are summarized in Table 3.

D1008030 (Ctrl 30) (unaffected)

D1008031 (Case 31) (affected)

D1008032 (Case 32) (affected)

D1008033 (Ctrl 33) (unaffected)

Brachspira NADH Oxidase 1 Gene (nox1) Assay

PCR analysis using primers Bnoxf (5′-TAG CYT GCG GTA TYG CWC TTT GG-3′) (SEQ ID NO:1) and BnoxR (5′-CTT CAG ACC AYC CAG TAG AAG CC-3′) (SEQ ID NO:2). This assay is based on Rohde et al. (1) and results in amplification of a 939 bp fragment of the Brachyspira NADH oxidase 1 gene (nox1). Primers have been demonstrated to amplify this fragment from B. hyodysenteriae, B. pilosicoli, B. intermedia, B. murdochii and B. innocens.

### Sequence Alignment

A multiple sequence alignment of 810 bp of the PCR product sequence with published Brachyspira nox1 sequences demonstrated that the pig isolate was distinct from published sequences, with a maximum pairwise DNA identity of 92.3%. Phylogenetic analysis was performed based on this nucleotide sequence analysis and on the translated peptide sequences (FIGS. 2A and B).

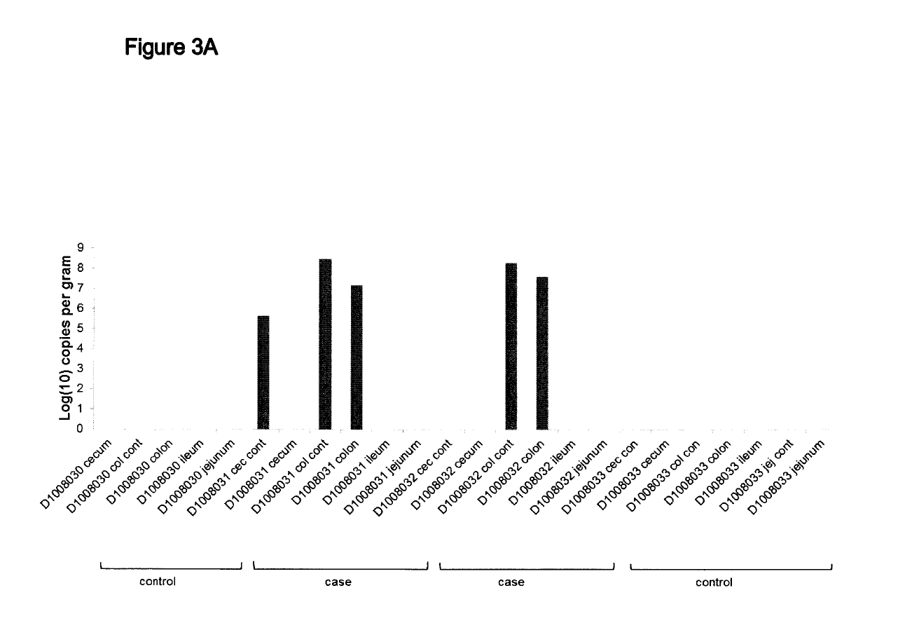

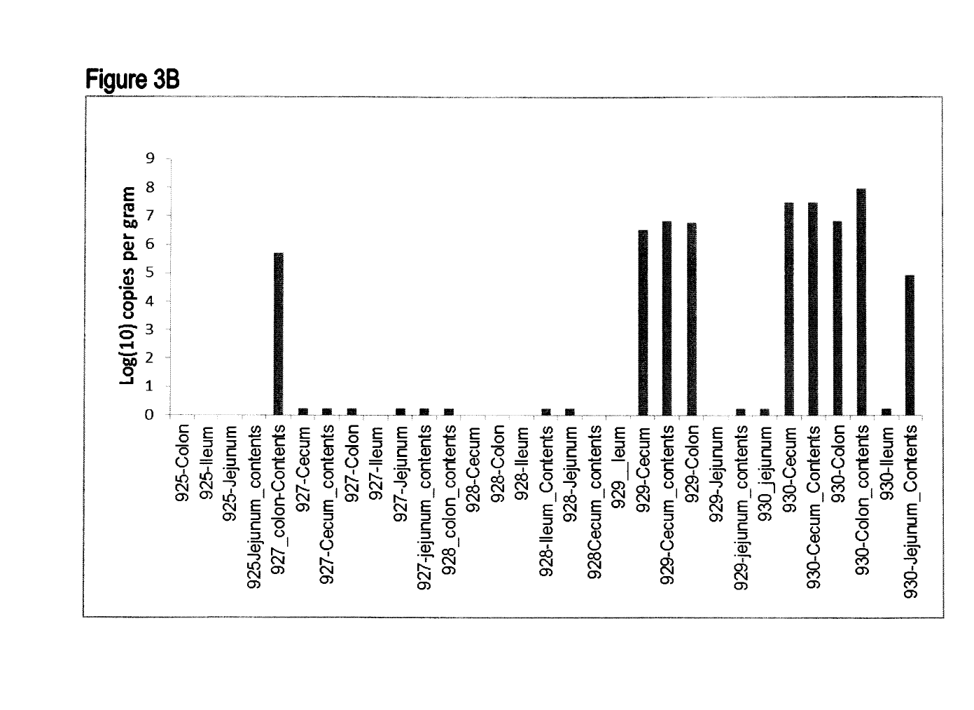

The analysis suggests that intra-species distances for nox1 are generally small and that the pig isolate is indeed a distinct entity in these trees. It's notable that one strain of B. intermedia (AF060810 Serpulina intermedia strain 2818.5) is an exception to the tight species clustering observed in all other species. Also, it's relative position in the nox1 phylogeny shifts markedly when peptide sequences are considered. This could relate to the original identification of the isolate and the observation of some species mixtures in the original publication (1). The distance of the case isolate from other Brachyspira spp. is consistent with this isolate representing a novel member of the genus. Recently, the JH0224/JH0225 assay was validated on a quantitative real-time PCR platform (SYBR green detection). The samples described above have been analysed using this qPCR assay and as expected, the greater sensitivity of the SYBR green detection results in the detection of the target organism in additional samples, albeit at lower levels. There appears to be a strong relationship between clinical symptoms and the relative abundance of the organism in tissue and contents (FIG. 3).

### Discussion

Swine dysentery is rare in western Canada. These cases of mucohaemorrhagic typhlocolitis were remarkable, given that neither Bh and Bp could be identified in tissue or feces of affected pigs. Although associated with colitis, we do not know if Brachyspira sp. Sask30446 is causal, but it is clearly genetically different from the known Brachyspira spp. infecting swine. It is possible that Brachyspira sp. Sask30446 is a normal inhabitant of the pig's gut, and under some situations, propagates and colonizes the colon resulting in pathology and clinical disease. Reports from affected farms suggests that diarrhea associated with Brachyspira sp. Sask30446 responds quickly to feed-grade antimicrobials, but diarrhea recurs when withdrawn. More studies are planned to determine its epidemiology and causality, but diagnostic labs and veterinarians should be aware that not all cases of mucohaemorrhagic diarrhea in swine appear to be associated with B. hyodysenteriae.

Atypical Brachyspira spp associated with colitis have been reported (Thomson et al 1998; Thomson et al 2001), but neither paper provided detailed genomic information on the specific strains.

### Materials and Methods

Tissue samples were weighed prior to DNA extraction to facilitate quantification. Genomic DNA was extracted from tissue samples using the Blood & Tissue Extraction Kit (Qiagen) according to manufacturer's instructions. Genomic DNA was extracted from feces or intestinal contents using the QIAamp DNA Stool Mini Kit (Qiagen). All assay reaction mixtures consisted of 1× iQ SYBR green supermix (Bio-Rad), 400 nmol/L concentrations of each of the appropriate primers, and 2 μL of template DNA in a final volume of 25 μL. An iCycler or MyiQ thermocycler (Bio-Rad) was used for all reactions with the following program: 95° C. for 3 min, followed by 40 cycles of 15 s at 95° C., 15 s at the appropriate annealing temperature, and 15 s at 72° C. A final melt at 95° C. for 1 min was done prior to a dissociation curve analysis (55° C. to 95° C. in 0.5° C. steps for 10 s increments). Fluorescence signals were measured every cycle at the end of the annealing step and continuously during the dissociation curve analysis. The resulting data were analyzed using iQ5 optical system software (Bio-Rad). All reactions were performed in duplicate (within the assay). A standard curve consisting of a 10-fold dilution series of plasmid containing the target DNA sequence was included with each set of samples analysed. Results were reported as target copy number per gram of tissue or feces.

### Example 2

### Additional Details Relating to Example 1

### Brachyspira sp. Isolated from Pigs

### History

A fibrinous, haemorrhagic mucopurulent colitis causing a clinical bloody diarrhea and death in finisher pigs (13 weeks or older) has been noted in up to 3 intensive finisher herds in Saskatchewan. Similar clinical signs have been noticed in a finisher herd(s) in Iowa that receive pigs from one of these Saskatchewan herds. Multiple diagnostic submissions, by the herd health veterinarians (summarized below) did not identify Brachyspira hyodysenteriae or B. pilosicoli in spite of similarities in terms of clinical expression, gross and histopathologic lesions.

### Farm D:

June 30 (D09-17757): Fresh and fixed tissues submitted from 23 week old pig. Histology—catarrhal colitis; intestinal tissue negative for B. hyodysenteriae and B. pilosicoli. August 26 (D09-22958): Fresh and fixed tissues submitted from 84 day old pig. Histology—colitis; intestinal tissue negative for B. hyodysenteriae and B. pilosicoli.

October 28 (D09-28573): Fresh and fixed tissues submitted from 84 day old pig with severe bloody diarrhea. Histology—necrotizing subacute diffuse mucoid colitis; intestinal tissue reported positive for B. murdochii.

### Farm H:

October 2 (D09-26620): Feces & Swabs submitted from multiple pigs. Negative for B. pilisicoli and B. hyodysenteriae and Lawsonia intracellularis.

October 8 (D09-27021): Swab and feces submitted. Species-specific PCRs negative for Brachyspira. Nox1 RFLP reported positive for B. intermedia, negative Lawsonia intracellularis

October 26 (D09-28403, -28404, -28405): On farm necropsy of 3 animals with bloody diarrhea, immediately fixed sections of colon and fecal swabs submitted. Histology indicative of swine dysentery (chronic mucopurulent colitis, subacute mucopurulent colitis, subacute fibrinosuppurative colitis). Gut swab negative for Brachyspira sp. on PCR. No Brachyspira seen on histo.

November 13 (D09-300446): Post Mortem lab with swine veterinary group. Eight suspect animals found dead overnight were necropsied by a swine practitioners visiting WCVM. Gross lesions were consistent with previous findings (fibrinous mucohaemorrhagic colitis/typhlitis with superficial necrosis). Histology not performed due to the lengthy time between death and sampling. Ligated sections of spiral colon submitted for culture (case #R09-138).

### Genomic Analysis of R09-138

Two isolates were obtained and PCR was performed by using primers Bnoxf (5′-TAG CYT GCG GTA TYG CWC TTT GG-3′) (SEQ ID NO:1) and BnoxR (5′-CTT CAG ACC AYC CAG TAG AAG CC-3′) (SEQ ID NO:2). This assay is based on Rohde et al. (2002) and results in amplification of a 939 bp fragment of the Brachyspira NADH oxidase 1 gene (nox1). Primers have been demonstrated to amplify this fragment from B. hyodysenteriae, B. pilosicoli, B. intermedia, B. murdochii and B. innocens.

The second step in the diagnostic assay is detection of restriction fragment length polymorphisms. This result for these particular isolates was inconclusive so PCR product generated from samples R09-138 #1&4 and R09-138 #3 were submitted for direct DNA sequencing using the amplification primers.

Identical sequences were obtained from the two PCR products.

A multiple sequence alignment of 810 bp of the PCR product sequence with published Brachyspira nox1 sequences demonstrated that the pig isolate was distinct from published sequences, with a maximum pairwise DNA identity of 92.3%. Phylogenetic analysis was performed based on this nucleotide sequence analysis and on the translated peptide sequences (see FIG. 2).

The phylogenetic trees of FIG. 2 are each the consensus of 100 neighbor-joined trees. Distance matrices for nucleotide sequence were calculated with dnadist using the Jukes-Cantor correction. Protein distances were calculated using the PAM matrix in protdist. Branch lengths were imposed on consensus dendrograms using fitch.

The analysis suggests that intra-species distances for nox1 are generally small and that the pig isolate is indeed a distinct entity in these trees. It's notable that one strain of B. intermedia (AF060810 Serpulina intermedia strain 2818.5) is an exception to the tight species clustering observed in all other species. Also, it's relative position in the nox1 phylogeny shifts markedly when peptide sequences are considered. This could relate to the original identification of the isolate and the observation of some species mixtures in the original publication (Rohde et al., 2002).

The distance of the case isolate from other Brachyspira spp. is consistent with this isolate representing a novel member of the genus.

B. suanatina was originally isolated from pigs and mallard ducks. This strongly haemolytic, indole positive spirochete is genetically distinct from B. hyodysenteriae and has been shown to cause swine dysentery-like disease in experimentally inoculated pigs (Rasback et al., 2007a; Jansson, 2009). This suggests that there are other Brachyspira-like organisms other than the “usual suspects” that can cause disease in pigs.

Based on the 810 bp alignment of Bnox sequences, PCR primers were designed (using SigOligo) that should amplify a 241 bp fragment of the Brachyspira sp. Sask30446 sequence.

(SEQ ID NO: 1)
BnoxF 5′-TAG CYT GCG GTA TYG CWC TTT GG-3′
[JH0222]
(SEQ ID NO: 2)
BnoxR 5′-CTT CAG ACC AYC CAG TAG AAG CC-3′
[JH0223]

New primers (below) predicted Tₘ is 60° C.).

(SEQ ID NO: 3)
5′-TCG CTA AAT TAT TCC AAC AAG GA-3′ [JH0224]
(SEQ ID NO: 4)
5′-AAA CGC ATT TCT ATT CCA GCA-3′ [JH0225]

### PCR

### Samples

D0930446 (2 extracts)

D0926620 (2 extracts)

D0927021

D0928403

D0928573

PCR was performed on these extracts using the BnoxF/BnoxR primers and the new JH0224/JH0225 pair. Bnox product was obtained from all except D0928403. JH0224/JH0225 PCR product was obtained from all cases.

The sequences of these amplified products are all identical to each other and to the D0930446 sequence shown in the phylogenetic tree. The sequence data also confirmed the specificity of the JH0224/JH0225 primer set, which could be applied to fixed tissue samples since the amplicon size is small.

Four pigs from an affected barn were brought for necropsy and full diagnostic work-up. We received samples of GI tract (jejunum, ileum, colon and contents) from all pigs. Total DNA extraction performed on all tissues and some contents for PCR. Samples were blinded to PCR operator.

D1008030 (unaffected)

D1008031 (affected)

D1008032 (affected)

D1008033 (unaffected)

Cultures were prepared from all materials. 11 culture lysates from a case from the JH case and some of the another case (pigs D1008031 and D1008032) (selected spirochete colonies boiled in PBS) for PCR to determine if these cultures contained the organism of interest. The JH case is from an additional farm, different than Farm D or Farm H.

PCR was performed on all lysates with primers JH0224/JH0225. Six of eleven were positive. Several were purified and sequenced. Sequences were about 99% identical and are further described as described in Example 3.

### PCR Results on Isolates (Primers JH0224/JH0225).

| Lysate label | PCR result | Sequence |
|---|---|---|
| R10-011-5A | + | confirmed |
| R10-011-5B # β hem | + | confirmed |
| R10-011-5A # H11 | − | ND |
| R10-011-5B #9 β hem | + | Confirmed |
| R10-011-5B # 10 H11 | + | ND |
| R10-011-5A # 5 β hem | + | ND |
| R10-011-5A # 3 β hem | + | Confirmed |
| 32-8-colon | − | ND |
| 12-32-jejunum | − | ND |
| 4-33-colon | − | ND |
| 11-30-colon | − | ND |

ND = not done.

### PCR Results on Tissue and Contents.

| | Ileum | Jejunum | | |
|---|---|---|---|---|
| | (tissue/ | (tissue/ | Cecum | Colon |
| Pig | contents) | contents) | (tissue/contents) | (tissue/contents) |
| D1008030 | +/− | −/ND | −/− | −/− |
| D1008031 | −/ND | −/ND | −/− | +/+ |
| D1008032 | −/− | −/ND | +/− | +/+ |
| D1008033 | −/− | −/− | −/− | −/− |

ND = not done (no contents recovered)
All of the PCR positives were sequenced and confirmed to be identical to anticipated target (data received 2010-04-29).

All of the PCR positives were sequenced and confirmed to be identical to anticipated target.

### Example 3

A 241 base pair sequence is amplified using primers SEQ ID NO: 3 and 4. This 241 base pair sequence has been amplified in several different isolates. The isolates share about 99% identity over the 241 base pair sequence. For example, SEQ ID NO:7 and SEQ ID NO:8 differ at positions 11 and 12.

SEQ ID NO: 7
>TM25_H02_2478-cnf_002 to.0.2
TCGCTAAATTTATCCAACAAGGACAGGATATCATTAATGAAATAGCTAAA
CCTGAAGTAAAAAAAGTTATGGTTGTTGGTGCTGGTTATATAGGTGTTGA
GCTTATAGAAGCATTCAAAAACCATGGTAAAGAAGTTATCTTAATGGAAT
CTATGCCTAGAGTTATGGCTAACTATTTTGATAAAGAAATCACTGATGAA
GCTGAAAAAAGAATCATAGATGCTGGAATAGAAATGCGTTT
SEQ ID NO: 8
>TM25_H06_2482-cnf_034 to.0.2
TCGCTAAATTATTCCAACAAGGACAGGATATCATTAATGAAATAGCTAAA
CCTGAAGTAAAAAAAGTTATGGTTGTTGGTGCTGGTTATATAGGTGTTGA
GCTTATAGAAGCATTCAAAAACCATGGTAAAGAAGTTATCTTAATGGAAT
CTATGCCTAGAGTTATGGCTAACTATTTTGATAAAGAAATCACTGATGAA
GCTGAAAAAAGAATCATAGATGCTGGAATAGAAATGCGTTT

### Example 4

Other primer pairs that specifically amplify NADPH oxidase include:

(SEQ ID NO: 5)
JH0249 5′-TCATAGATGCTGGAATAGAAATGC-3′
(SEQ ID NO: 6)
JH0250 5′-GCACCGTTAGGTAAAGTTTCCA-3′

These amplify a 184 bp region of the Bnox sequence. The optimal annealing temperature is 63 C. The primer pair was tested for PCR efficiency using a plasmid-based standard curve.

Table of Sequences
Primers

NADH oxidase 1 gene (nox1) of Brachyspira spp.
Bnoxf (5′-TAG CYT GCG GTA TYG CWC TTT GG-3′) (SEQ ID NO: 1)
BnoxR (5′-CTT CAG ACC AYC CAG TAG AAG CC-3′) (SEQ ID NO: 2)
Where W = A or T and Y = C or T
Brachyspira sp. Sask30446 - 241 bp amplification product
5′-TCG CTA AAT TAT TCC AAC AAG GA-3′ [JH0224] (SEQ ID NO: 3)
5′-AAA CGC ATT TCT ATT CCA GCA-3′ [JH0225] (SEQ ID NO: 4)
Brachyspira sp. Sask30446 - 184 bp amplification product
JH02495′-TCATAGATGCTGGAATAGAAATGC-3′ (SEQ ID NO: 5)
JH02505′-GCACCGTTAGGTAAAGTTTCCA-3′ (SEQ ID NO: 6)
Brachyspira sp. Sask30446 NADPH oxidase polynucleotide sequences
SEQ ID NO: 7
>TM25H02_2478-cnf_002 to.0.2
TCGCTAAATTTATCCAACAAGGACAGGATATCATTAATGAAATAGCTAAACCTGAAGTAA
AAAAAGTTATGGTTGTTGGTGCTGGTTATATAGGTGTTGAGCTTATAGAAGCATTCAAAA
ACCATGGTAAAGAAGTTATCTTAATGGAATCTATGCCTAGAGTTATGGCTAACTATTTTG
ATAAAGAAATCACTGATGAAGCTGAAAAAAGAATCATAGATGCTGGAATAGAAATGCGTTT
SEQ ID NO: 8
>TM25_H06_2482-cnf_034 to.0.2
TCGCTAAATTATTCCAACAAGGACAGGATATCATTAATGAAATAGCTAAACCTGAAGTAA
AAAAAGTTATGGTTGTTGGTGCTGGTTATATAGGTGTTGAGCTTATAGAAGCATTCAAAA
ACCATGGTAAAGAAGTTATCTTAATGGAATCTATGCCTAGAGTTATGGCTAACTATTTTG
ATAAAGAAATCACTGATGAAGCTGAAAAAAGAATCATAGATGCTGGAATAGAAATGCGTTT
Brachyspira sp. Sask30446 Bnox (810 nucleotides) (SEQ ID NO: 9)
CCTGAAGGTTTGAAATCTGAAGGCATCGATGTTTATATGGGACATGAAGTAACAAAAATAGACTGGGC
TAACAAAAAATTACATATCAAAGAATTAAAAACAGGTAAAGAGTTTGAAGACAATTATGATAAACTTA
TACTTGCTACTGGTTCTTGGCCTGTAACTCCTCCTATAGAAGGATTAAAACAAGAAGGTACTGAATAC
GGTCTTAAAAAAGGTATTTTCTTCGCTAAATTATTCCAACAAGGACAGGATATCATTAATGAAATAGC
TAAACCTGAAGTAAAAAAAGTTATGGTTGTTGGTGCTGGTTATATAGGTGTTGAGCTTATAGAAGCAT
TCAAAAACCATGGTAAAGAAGTTATCTTAATGGAATCTATGCCTAGAGTTATGGCTAACTATTTTGAT
AAAGAAATCACTGATGAAGCTGAAAAAAGAATCATAGATGCTGGAATAGAAATGCGTTTAGGTGAAAC
TGTTAAAAAGTTTGAAGGTGATGACAGAGTTAAAAAAGTTGTTACCGATAAAGGTTCTTATGATGTAG
ATATGGTAGTTATGTCTGTTGGTTTCAGACCTAATAGCGAACTTTATAAAGACTATTTGGAAACTTTA
CCTAACGGTGCTATTGTAGTAGATACTACTATGAGATCAAGCAAAGATCCTGATGTTTATGCTATAGG
AGACTGTGCTACTGTATATTCAAGAGCTTCTGAAAAACAAGAGTATATTGCTTTAGCTACTAATGCTG
TAAGAATGGGTATTGTTGCTGCTAATAATGCTTTAGGAAAACATGTTGAATACTGCGGTACT
Nearest DNA relative is 92.2% identical to:
EF517546 Brachyspira innocens isolate C336 (SEQ ID NO: 10)
CCTGAAAGTTTAAAAGCTGAAGGCATAGATGTTTATATGGGACATGATGTAACAAAAATAGACTGGGC
TAATAAAAAATTACATGTTAAAGAATTAAAAACAGGTAAAGAGTTTGATGATAATTATGACAAACTTA
TTCTTGCTACAGGTTCTTGGCCTGTAACTCCTCCTATAGAAGGTTTAATGCAGGAAGGTACTGAATAC
GGACTTAAAAAAGGTATTTTCTTCTCTAAATTATTCCAGCAAGGACAAGAAATTATTGATGAAATAGC
TAAACCTGAAGTAAAAAAAGTTATGGTAGTTGGTGCTGGTTATATAGGTGTTGAACTTATAGAAGCTT
TCAAAAATCATGGTAAAGAAGTTATATTAATGGAAGCTATGCCTAGAGTTATGGCTAACTATTTTGAC
AAAGAAATTACTGATGAAGCTGAAAAAAGAATCAAAGAAGCTGGCATAGAAATGCATTTAGGTGAAAC
TGTTAAAAAATTTGAAGGTGATGACAGAGTTAAAAAAGTTATTACTGACAAAGGTTCTTATGATGTAG
ATATGGTAGTTATGTCTGTTGGTTTCAGACCTAATAGTGAGCTTTATAAAGATTATTTAGAAACTTTA
CCTAATGGTGCTATAAAAGTAGACACTACTATGAAAACTACAAAAGATCCTAATGTATTTGCTATAGG
AGACTGTGCTACTGTATATTCAAGAGCTTCTGGAAAAGAAGAATACATCGCTTTAGCTACTAATGCTG
TAAGAATGGGATTGTTGCTGCTAACAATGCTTTAGGAAAACATGTTGAATACTGCGGTACT
>translation of 241 bp fragment from Brachyspira sp. Sask30446
(SEQ ID NO: 11)
AKFIQQGQDIINEIAKPEVKKVMVVGAGYIGVELIEAFKNHGKEVILMESMPRVMANYFDKEIT
DEAEKRIIDAGIEMR
>810 bp translation from Brachyspira sp. Sask30446 SEQ ID NO: 12
PEGLKSEGIDVYMGHEVTKIDWANKKLHIKELKTGKEFEDNYDKLILATGSWPVTPPIEGLKQ
EGTEYGLKKGIFFAKLFQQGQDIINEIAKPEVKKVMVVGAGYIGVELIEAFKNHGKEVILMESM
PRVMANYFDKEITDEAEKRIIDAGIEMRLGETVKKFEGDDRVKKWTDKGSYDVDMVVMSVG
FRPNSELYKDYLETLPNGAIVVDTTMRSSKDPDVYAIGDCATVYSRASEKQEYIALATNAVRM
GIVAANNALGKHVEYCGT
>AAC78822 NADH oxidase [Brachyspira murdochii DSM 2563] (SEQ ID NO: 13)
PESLKAEGIDVYMGHDVTKIDWANKKLHVKELKTGKEFDDNYDKLILATGSWPVTPPIEGLM
QEGTEYGLKKGIFFSKLFQQGQEIIDEIAKPEVKKVMVVGAGYIGVELIEAFKNHGKEVILMEA
MPRVMANYFDKEITDEAEKRIKEAGIEMHLGETVKKFEGDDRVKRVVTDKGSYDVDMVVMS
VGFRPNSELYKDYLETLPNGAIKVDTTMKTTKDPNVFAIGDCATVYSRASGKEEYIALATNAV
RMGIVAANNALGKHVEYCGT

Brachyspira murdochii and B. innocens are the closest related sequence to the translation product of the 810 Brachyspira sp. Sask30446 nox1 sequence and are 91% identical (SEQ ID NO:13) and 92-93% identical respectively, depending on the strain.

>AAC78816 NADH oxidase [Brachyspira pilosicoli]
(SEQ ID NO: 14)
SKLYQQGQDIINEIAKPEVKKVMVVGAGYIGVELIEAFKNHGKEVILMEAMPRVMANYFDKEIT
DEAEKRIKDAGIELH

The above species is 91% identical to B. Sask30446 NADPH oxidase at amino acid level which is the closest reference sequence for this region of amino acid sequence.

### Example 5

### Diagnostic Method for Colitis

### DNA Sequence-Based Identification of Brachyspira sp. Sask30446

In addition to the NADPH oxidase (nox) sequence-based identification described previously, the DNA sequence of several other genes in the genome of Brachyspira sp. Sask30446 have been determined. All sequences presented here were derived from cultured isolates (see Example 7) and based on currently available sequence data for Brachyspira spp., all of these sequences can be used to discriminate Brachyspira sp. Sask30446 from other recognized species in the genus Brachyspira, e.g. they are diagnostic for Sask30446.

Sequences for chaperonin-60 and 16S rRNA were determined as described below, using established primers and PCR protocols to amplify the target regions from isolates.

A Multi Locus Sequence Typing (MLST) scheme originally published for the identification and strain differentiation of Brachyspira spp. by Rasback et al. (2007b) was applied to isolates of Brachypira sp. Sask30446. Six samples from three separate culture plates were examined. In all cases, identical sequences were obtained for each of the targets. Representative sequences for each target are included, along with protein translations of the relevant open reading frames.

TABLE 4
Primers used in MLST (Rasback et al., 2007b)

| | PRIMER | |
|---|---|---|
| TARGET | NAME | SEQUENCE (5′-3′) |
| esterase (est) | EST-F229 | GATGCTTCAGGCGGAGTTATG |
| | | (SEQ ID NO: 15) |
| | EST-R847 | CCACACTCATAGCATAAATACTG |
| | | (SEQ ID NO: 16) |
| glucose kinase | GLPK- | AGGCTGGGTAGAACATAATGC |
| (glpk) | F123 | (SEQ ID NO: 17) |
| | GLPK- | TCTTTACTTTGATAAGCAATAGC |
| | R1158 | (SEQ ID NO: 18) |
| phosphoglucomutase | PGM- | GTTGGTACTAACAGAATGAATA |
| (pgm) | F172 | (SEQ ID NO: 19) |
| | PGM- | CCGTCTTTATCGCGTACATT |
| | R1220 | (SEQ ID NO: 20) |
| acetyl-CoA | THIO- | TGTGTTATACAATCAGCACTTC |
| acetyltransferase | F163 | (SEQ ID NO: 21) |
| (thioloase)(thi) | THIO- | GTAGTAAGTATTCTAGCTCCAG |
| | R1079 | (SEQ ID NO: 22) |

chaperonin-60 (cpn60 or GroEL or hsp60)

Target sequence corresponds to the cpn60 “universal target”, widely exploited for bacterial species identification (Hill et al., 2004) (e.g. universal, degenerate PCR primers which can be applied for the amplification of a 549-567 bp region of cpn60 corresponding to nucleotides 274-828 of the E. coli cpn60 sequence from virtually any genome). Sequence was amplified from an isolated colony using primers H729 (5′-CGC CAG GGT TTT CCC AGT CAC GAC GAI III GCI GGI GAY GGI ACI ACI AC-3′) (SEQ ID NO:23) and H730 (5′-AGC GGA TAA CAA TTT CAC ACA GGA YKI YKI TCI CCR AAI CCI GGI GCY TT-3′) (SEQ ID NO:24) and published PCR conditions (Brousseau et al., 2001).

>v12564 Brachyspira sp. Sask30446 chaperonin-60 (cpn60)
(SEQ ID NO: 25)
GCTACTGTTTTAGCTCAGGCTATGGTTAAAGAAGGTTTGAAAAATGTAACTAGCGGTGCT
AATCCTATGCTTATTAAAAGAGGTATAGAAAAAGCTGTTAGCGAAATAGTTGCTTATATC
AAATCTGAAGCTAAACAAATTAAAGGCAAAGAAGAAATTGCTCAGGTTGCTACTATTTCT
GCTAACAATGATAAAGAAATTGGTGCTTTAATCAGTGATGCTATGGAAAAAGTTGGTAAA
GAAGGCGTTATCACTGTAGAAGAAGCTAAATCTTTAGAAACTAGTCTTTCTTTGGTAGAA
GGTATGCAGTTTGACAGAGGTTATATCTCTCCATATTTCGTAACTAACGGAGATAGTATG
ACTGCTGAATTAGAAGATGCTTTACTTCTTATCTATGATAAAAAAATCTCTAACATGAAAG
AACTTCTTCCTATACTTGAAAAAATTGCTCAGACTGGAAGACCTTTCATTATTATCGCTGA
AGATATTGAAAATGAAGCTTTGGCTACTTTGGTACTTAACAAAATGAGAGGAGTATTAAAT
GTATGTGCTGTT

This nucleotide sequence is 97% identical to Brachyspira intermedia (Genbank Accession JF907595), 96% identical to Brachyspira murdochii DSM 12563 (Genbank Accession CP001959), 96% identical to Brachyspira murdochii ATCC 51284 (Genbank Accession DQ099908), 95% identical to Brachyspira innocens ATCC 29796 (Genbank Accession DQ099906), 94% identical to Brachyspira hyodysenteriae WA1 (Genbank Accession CP001357), 94% identical to Brachyspira hyodysenteriae ATCC 27164 (Genbank Accession DQ099905), 92% identical to Brachyspira pilosicoli ATCC 51139 (Genbank Accession DQ099903), 91% identical to Brachyspira alvinipulli ATCC 51933 (Genbank Accession DQ099907), 84% identical to Brachyspira aalborgi ATCC 43994 (Genbank Accession DQ099904). The nucleotide sequence encodes the following peptide sequence (reading frame+1):

>v12564 Brachyspira sp. Sask30446 chaperonin-60 (cpn60)
(SEQ ID NO: 26)
ATVLAQAMVKEGLKNVTSGANPMLIKRGIEKAVSEIVAYIKSEAKQIKGKEEIAQVATISANND
KEIGALISDAMEKVGKEGVITVEEAKSLETSLSLVEGMQFDRGYISPYFVTNGDSMTAELEDA
LLLIYDKKISNMKELLPILEKIAQTGRPFIIIAEDIENEALATLVLNKMRGVLNVCAV

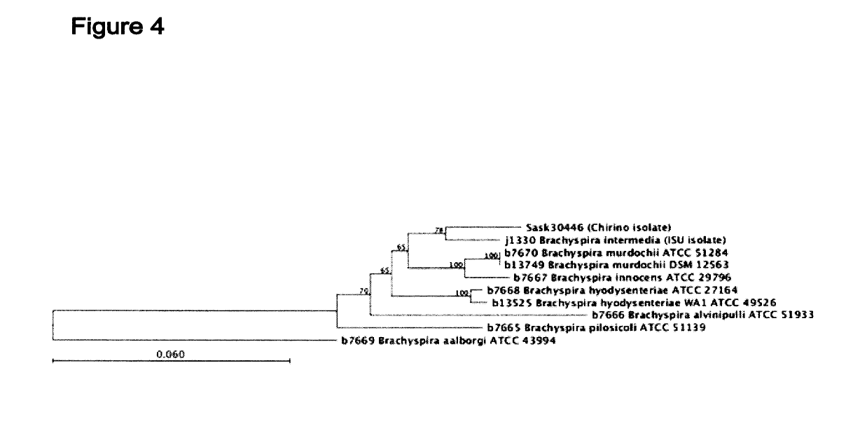

The nearest peptide neighbour is B. hyodysenteriae at 98% identity. A phylogenetic tree showing the relationship of Brachyspira sp. Sask30446 to other species, based on cpn60 universal target sequence (555 bp) is shown in FIG. 4.
Esterase (est)
DNA sequence of Sask30446 est MLST target:

>Brachyspira sp. Sask30446 esterase (est)
(SEQ ID NO: 27)
AAATATTTAGAAGAGAGAAATATAGGTTTTGATGTTGGAGTTACAAAGGTTCCTTTGGTTT
GTCAGTCTTGTATATTTGATTTGCGTGTTGGGGATTATAAAGTTCGTCCGGATATTAATAT
GGCTTATGAGGCTTGTATCAATGCTCAAAATAATAATCCAAAAATGGGAAATTATGGAGC
TGGTACAGGTGCTAGTGTTGGAAAAATACTTGGTGCTGATTATACTATGAAGTCTGGGCT
TGGTTTTTATGCTGTTCAAATTGATGATGTTAAGGTTGGTGCTATAGTTGCTCTTAATGCT
TTCGGCGATATTTATGATTATGATAATGGAAAAATGATTGCTGGTCTTCTTAATGAAAATA
AAGATGGGTTTAGAAGTTCTGAAGAGGAGCTTATAAAAATAACGCAGAATAATAATTTGT
CTTTTACTTCTAATAATAATATAGTTACAAATACTACAATAGGAGCTGTAATTACAAATGCC
AAGTTTACAAAGTCACAAATGGGAAAAATAGCTTCTATGGCACATAACGGCTTTGCTAGG
GCTATAAAACCAGTGCATACTACAGTAGATGGCGA

This DNA sequence is 93% identical to the est sequence of Brachyspira murdochii DSM12563 (Genbank Accession CP001959), 86% identical to Brachyspira hyodysenteriae WA1 (Genbank Accession CP001357). This nucleotide sequence encodes the following protein sequence (Reading frame+1):

>Brachyspira sp. Sask30446 esterase (est)
(SEQ ID NO: 28)
KYLEERNIGFDVGVTKVPLVCQSCIFDLRVGDYKVRPDINMAYEACINAQNNNPKMGNYG
AGTGASVGKILGADYTMKSGLGFYAVQIDDVKVGAIVALNAFGDIYDYDNGKMIAGLLNE
NKDGFRSSEEELIKITQNNNLSFTSNNNIVTNTTIGAVITNAKFTKSQMGKIASMAHNGF
ARAIKPVHTTVDG

The nearest peptide neighbour is B. murdochii at 95% identity.
Glucose Kinase (glpk)
DNA sequence of Sask30446 glpk MLST target:

>Brachyspira sp. Sask30446 glucose kinase (glpk)
(SEQ ID NO: 29)
GGAGTTTGCAGGAGCTATACAGATTGCTGGAGTTAAGCCTGAAGAGATAGCTGCTATTG
GTATTACAAATCAAAGGGAAACTACTGTTGTATGGGATAAAAATACTGGAGAGCCTATTT
ATAATGCTATAGTATGGCAATGTAGAAGAACTGCTCCTATTTGCGATGATTTAAAGAAAAA
AGGACTTGATACTTATATAAGAGAAAATACAGGTTTAGTTGTTGATGCTTATTTTTCTGGT
ACAAAAATAAAATGGATACTTGATAATGTTCCTGGTGCTAGAGAAAAAGCCAATAAAGGA
GAATTACTATTTGGTACAATAGACACTTGGCTTGTATGGAAACTTACAGGCGGTAAAGTT
CATGTTACAGACTATACTAATGCATCAAGGACTATGATTTATAATATCAAAGATTTAAAAT
GGGACGAAAACATTTTAAGAGAATTAGACATTCCTATGAGCATGCTTCCAGAGGTAAAAG
ATTCTTCTTGTGTTTACGGATATGCTAATATTAACGGTAAAGAAGTACCTATATCTGGAAT
AGCAGGAGACCAGCAGTCAGCATTATTCGGTCAGGCTGGATTTAATAAGGGCGACACAA
AAAATACTTATGGTACTGGAAGTTTCATTCTTATGAATATAGGAGAGAATTTTATATTAAGT
AAAAACGGACTTATTACTACTATAGGTATAGGATATAATGGAAAAATAGAATATGCTTTGG
AAGGTTCTGTATTTATAGCTGGTGCTGTTATACAATGGGTACGCGATGAATTAAGACTTC
TTCATGATGCAAAAGACACTGAATATTTCGCTACTAAAGTAAAAGATACTAACGGAGTTTA
TTTAGTACCTGCATTTGTTGGTCTTGGTGCTCCTTATTGGGATATGTATGCTAGAGGCTG
TTTAGTAGGTATTACAAGAGGGGT

This sequence is 95% identical to Brachyspira murdochii DSM 12563 (Genbank Accession CP001959), 90% identical to Brachyspira hyodysenteriae WA1 (Genbank Accession CP001357) and 89% identical to Brachyspira pilosicoli strain 95/1000 (Genbank Accession CP002025). The nucleotide sequence encodes the following protein sequence (reading frame+2):

>Brachyspira sp. Sask30446 glucose kinase (glpk)
(SEQ ID NO: 30)
EFAGAIQIAGVKPEEIAAIGITNQRETTVVWDKNTGEPIYNAIVWQCRRTAPICDDLKKK
GLDTYIRENTGLVVDAYFSGTKIKWILDNVPGAREKANKGELLFGTIDTWLVWKLTGGKV
HVTDYTNASRTMIYNIKDLKWDENILRELDIPMSMLPEVKDSSCVYGYANINGKEVPISG
IAGDQQSALFGQAGFNKGDTKNTYGTGSFILMNIGENFILSKNGLITTIGIGYNGKIEYA
LEGSVFIAGAVIQWVRDELRLLHDAKDTEYFATKVKDTNGVYLVPAFVGLGAPYWDMYARG
CLVGITRGV

The nearest peptide neighbour is B. murdochii at 99% identity.
Phosphoglucomutase (pgm)
DNA sequence of Sask30446 pgm MLST target:

>Brachyspira sp. Sask30446 phosphoglucomutase (pgm)
(SEQ ID NO: 31)
CAGGTTTAGCTAATTATATATTAAAACAAGGCGGAAGTGATTATAAAGTTGCTATAGGATA
TGATTCAAGAAATAATTCTGATGTATTTTCAAAAGCTGCTGCTGAGATACTTTCTTCAA
ACGGAATAAAAGTTTATTTATATGATGATATTCACCCTATTTCACTTCTTTCTTATGCTG
TTAGAAGTTTAGGCTGTATTGCAGGAATAGTTGTTACTGCTAGTCATAACCCTAAGGAAT
ATAATGGATATAAAGTTTATTGGACTGACGGAGCTCAGGTTATACCGCCTCATGACAAAA
ATATCATAGATGAAGTATTAAAAGTTAAGCCTGAAGAAGTAAAAATGGGCGACTCTTCAA
AAATTACTATAATAGGAAAAGATATTGAAGATAAATACATGAATGATTTGATGGTATATTTA
GTTAATCCAGACATCATTAAAAAACATCATGATATAAAAATAGTTTATACCCCAATTCATG
GTTCTGGATACAAAATGGTTCCTATGGCTTTAAGAAAGGCTGGTTTTACAAATTTAACAAC
ATTAGAAGGTGCTCAGCCTCCAGACGGAAATTTTCCTACTGTAGAATCTCCTAACCCAGA
AAATCCTGAAGCATTGCAGATAGCCGTTAATAAAGCTAAAGAGATAGGTGCAGAACTTGT
TATGGGTACCGACCCAGACTGCGACAGAATGGGATGTGCTTTGCTTACTAAAGATGGAA
GCTATATGTATCTTACAGGTAATCAGATAGGATCCATAATGGCATACTATCTCATCACAAA
CAAAAAAAATATTAAAAATCCGTACATAGTAAAAACAATAGTAACAACTGAACTAGCTAGG
GCTATTGCTGATGCTAATAATGTTAAGATTTACGATGTACTTACAGGTTTTAAATGGATTG
CTGATGTTATAGAAAGAGATAAAGAAGGTACATATTTAT

This sequence is 93% identical to Brachyspira murdochii DSM 12563 (Genbank Accession CP001959), 89% identical to Brachyspira hyodysenteriae WA1 (Genbank Accession CP001357) and 88% identical to Brachyspira pilosicoli strain 95/1000 (Genbank Accession CP002025). This nucleotide sequence encodes the following protein sequence (reading frame+3):

>Brachyspira sp. Sask30446 phosphoglucomutase (pgm)
(SEQ ID NO: 32)
GLANYILKQGGSDYKVAIGYDSRNNSDVFSKAAAEILSSNGIKVYLYDDIHPISLLSYAV
RSLGCIAGIVVTASHNPKEYNGYKVYWTDGAQVIPPHDKNIIDEVLKVKPEEVKMGDSSK
ITIIGKDIEDKYMNDLMVYLVNPDIIKKHHDIKIVYTPIHGSGYKMVPMALRKAGFTNLT
TLEGAQPPDGNFPTVESPNPENPEALQIAVNKAKEIGAELVMGTDPDCDRMGCALLTKDGS
YMYLIGNQIGSIMAYYLITNKKNIKNPYIVKTIVTTELARAIADANNVKIYDVLTGFKW
IADVIERDKEGTYL

The nearest peptide neighbour is B. murdochii at 99% identity.

### Acetyl-CoA Acetyltransferase (Thioloase)(thi)

DNA sequence of Sask30446 thi MLST target:

>Brachyspira sp. Sask30446 acetyl-CoA acetyltransferase (thioloase)(thi)
(SEQ ID NO: 33)
TATTAATGCCGGAATACCTGTAGACAAACCGGCATTAACTATAAATATATTATGCGGTTC
AGGATTAAGAGCTGTATCAATGGCAGCACAAATGATTAAATCAGGAGATGCTGATATAGT
AGTTGCAGGCGGTACTGAAAATATGAGTATGGCTCCATATACTTCTTCAGCTATGAGAAT
GGGTGCTAGAATGGGCGAAAGTAAAATGCAGGATACTTTACTTAATGATGCTCTTATTTG
TGCTTTTGAGCATTATCATATGGGAGTTACTGCTGAAAATGTTGCAGAACAATGGGGTAT
TACAAGACAAGAACAAGATGAGTTTGCATGCAGAAGCCAGAACAGAGCAGAAGAAGCAG
TAAAAAGCGGAAGATTTAAAGATGAAATAGTACCTGTTACAATCAAAACTAGAAAAGGG
AAATAGTAGTTGATACAGATGAACACCCTACTTTTGGTACTACTATGGAATCTTTAGCTAA
ATTAAAACCTGCATTCAAAAAAGATGGTACTGTTACTGCTGGTAATGCTTCTGGTATCAAT
GATGCTGCTTCAGCTGTAGTTATTATGTCTAAAGAAAAAGCTGATGAGCTTGGAATTAAA
CCTATGGCTAAGATTTTAGGTTATGCTACTCATGGTGTTGAGCCTAGAATAATGGGTATA
GGACCTATAGAGGCAACTAGAAAGGCTTTGAAAATGGCTAATCTTACAGTTGAAGATATG
GACTTAATAGAAAGTAATGAGGCTTTCGCTGCTCAGTCTATAGCTGTTGCCCGTGAGTTA
AAATTCAATATGGACATAGTTAATGTCAATGGCGGAGCTA

This nucleotide sequence is 88% identical to Brachyspira murdochii DSM 12563 (Genbank Accession CP001959), 92% identical to Brachyspira hyodysenteriae WA1 (Genbank Accession CP001357) and 88% identical to Brachyspira pilosicoli strain 95/1000 (Genbank Accession CP002025). This sequence encodes the protein sequence (reading frame+2):

>Brachyspira sp. Sask30446 acetyl-CoA acetyltransferase (thioloase)(thi)
(SEQ ID NO: 34)
INAGIPVDKPALTINILCGSGLRAVSMAAQMIKSGDADIVVAGGTENMSMAPYTSSAMRMGA
RMGESKMQDTLLNDALICAFEHYHMGVTAENVAEQWGITRQEQDEFACRSQNRAEEAVKS
GRFKDEIVPVTIKTRKGEIVVDTDEHPTFGTTMESLAKLKPAFKKDGTVTAGNASGINDAASA
VVIMSKEKADELGIKPMAKILGYATHGVEPRIMGIGPIEATRKALKMANLTVEDMDLIESNEAF
AAQSIAVARELKFNMDIVNVNGGA

The nearest neighbour is B. pilosicoli at 95% identity.
Small Subunit Ribosomal RNA (16S rRNA or SSU RNA)
Amplified sequence corresponds to nucleotides 11-536 of the E. coli 16S rRNA gene (encompassing variable regions V1, V2 and V3). Amplification primers were H1476 (5′-GAG TTT GAT CCT GGC TCA G-3′) (SEQ ID NO: 35) and H1478 (5′-GWA TTA CCG CGG CKG CTG-3′) (SEQ ID NO:36) (Dorsch and Stackebrandt, 1992).

>Brachyspira sp. Sask30446 16S rRNA, partial
(SEQ ID NO: 37)
AGCGAACGTTGGCGATGCGTCTTAAGCATGCAAGTCGAGCGGACTTATTCGGGCAACTG
GATAAGTTAGCGGCGAACTGGTGAGTAACACGTAGGTAATCTGCCGTAGAGTGGGGGA
TAACCCATGGAAACATGGACTAATACCGCATATACTCTTACTACACAAGTAGAGTAGAGG
AAAGGAGCAATCCGCTTTACGATGAGCCTGCGGCCTATTAGCCTGTTGGTGGGGTAACG
GCCTACCAAAGCTACGATAGGTAGCCGACCTGAGAGGGTGACCGGCCACATTGGGACT
GAGATACGGCCCAGACTCCTACGGGAGGCAGCAGCTGAGAATCTTCCACAATGGACGA
AAGTCTGATGGAGCGACATCGCGTGAGGGATGAAGGCCTTCGGGTTGTAAACCTCGGA
AATTATCGAAGAATGAGTGACAGTAGATAATGTAAGCCTCGGCTAACTACGTGC

This sequence, while not identical to any published 16S rRNA sequences, is 98-99% identical to several other Brachyspira spp. However, it is well-established that 16S rRNA sequences are insufficient to discriminate Brachyspira spp. The 16S rRNA sequences are widely used to describe new bacterial species, and was determined as part of the description of Brachyspira sp. Sask30446.

### Example 6

### Case-Control Submission (Farm L)

Five 50 kg pigs with (Cases, n=3) or without (Controls, n=2) bloody diarrhea were examined from Farm L. All pigs were brought to WCVM for necropsy and full diagnostic work-up. An acute mucohaemorrhagic typhlocolitis was found in all Case pigs consistent with FIG. 1. No enteritis or typhlocolitis was found in either Control. Brachyspira-like organisms were identified by direct examination of the colon and caecum in all 5 pigs, but the level of organisms identified per high power field was substantially higher in Case compared to Control pigs. Intestinal tissue from all pigs tested negative for Bh, Bp, Binn and Li by PCR, negative for porcine circovirus type 2 (PCV2) by IHC, and negative for rotavirus and transmissible gastroenteritis by FAT. PRRS virus was detected in serum of Control but not Case pigs. Salmonella sp. was cultured from pooled intestinal tissues (caecum, colon, ileum) from one unaffected (Control 925) and one affected (Case 929) pig.

Below is case-control data from an additional farm (Farm

A number of high power fields were examined and the number of Brachyspira-like organisms were counted in each field then averaged across all fields.

Grade 0: 0 bacteria present per microscopic field, negative sample
Few: between 0 and 1 bacterium present per microscopic field
Grade 1: 1 bacterium present per microscopic field
Grade 2: 2-4 bacteria present per microscopic field
Grade 3: 5-20 bacteria present per microscopic field
Grade 4: >20 bacteria present per microscopic field

| | Cases | | | Controls | |
|---|---|---|---|---|---|
| | Pig ID | | | | |
| | | | 930 | | |
| | 927 | 929 | (medicated) | 925 | 928 |
| Histologic lesions | Mucohaemorrhagic typhylocolitis | | | NAF | |
| Direct smear | Caecum: | Caecum: | Caecum: 2+ | Caecum: | Caecum: |
| (Brachy-like org) | 2+ | 3+ | Colon: 3+ | 1+ | few |
| | Colon: 2+ | Colon: 4+ | | Colon: 1+ | Colon: few |
| Salmonella (pooled GI | Neg | Pos | Neg | Pos | Neg |
| tissues) | | | | | |
| Rotavirus (FA: Ileum) | Neg | Neg | Neg | Neg | Neg |
| TGE (FAT: Ileum) | Neg | Neg | Neg | Neg | Neg |
| PRRS (PCR: Serum) | Neg | Neg | Neg | Pos | Pos |
| Bhyo, B pilo, Binn | Neg | Neg | Neg | Neg | Neg |
| Lawsonia (PCR: Ileum) | Neg | Neg | Neg | Neg | Neg |
| PCV2 (IHC: Ileum, jej, | Neg | Neg | Neg | Neg | Neg |
| caecum) | | | | | |

### Example 7

### Agar Culture Characteristics of Brachyspira sp. Sask30446

Brachyspira sp. Sask30446 is a fastidious, slow growing, anaerobe. Growth of Sask30446 has best been achieved on BAM-SR agar (Blood Agar Base no. 2 (Oxoid) (40 g l⁻¹), Beef Extract (Difco) (3 g l⁻¹) and Bacto Peptone (Difco) (5 g l⁻¹)), supplemented with defibrinated horse blood (7%), spectinomycin (400 mg ml⁻¹) and rifampin (30 mg ml⁻¹) (Calderaro et al., 2005). Growth (confirmed by a positive PCR result with Sask30446-specific assay) and Gram stain, has also been observed on this media made with sheep blood instead of horse blood, although isolated colonies were not obtained.

Direct inoculation of agar media with colon tissue from an affected pig.

Inoculation of agar media through a 0.45 μm filter (sample incubated on top of filter on top of media)

Inoculation of agar media with broth that was incubated with the tissue sample at room temperature for 30 minutes. Inoculation can be directly on to the agar surface or through a 0.45 micron filter.

After 8 days of incubation at 37° C., Brachyspira sp. Sask30446 is visible as a weakly hemolytic (on horse blood), tiny, clear, wet/glistening, “fried egg” colony. The colonies are less than 1 mm in diameter (FIG. 5).

Identity of cultured Sask30446 was confirmed by application of the Sask30446-specific nox-based PCR assay described previously and sequencing of the resulting PCR products to confirm their identity to the reference sequence.

Gram stains of isolates from agar plates show pleiomorphic, Gram negative spirochetes with tapered ends. Length of the cells is variable, 2-20 μm (FIG. 6). Spriochetes with the same morphology are abundant in samples of feces) from pigs with hemorrhagic colitis that also test positive by the Sask30446 qPCR assay (primers JH0224/JH0225) with counts of ˜1×10⁷ Sask30446 organisms per gram of feces or colon contents (FIG. 7).

### Example 8

For detecting Brachyspira sp. Sask 30446 or diagnosing a Brachyspira sp. Sask 30446 infection in a live animal, a fecal sample is obtained. For post-mortem diagnosis, colon tissue or colon contents are assayed. The species-specific nox-based primer set in either conventional or quantitative real-time PCR can be applied with SYBR green and the presence of the DNA is quantitated. Other gene targets can also be used. For example, PCR can used with the broad-spectrum primers to amplify any other Brachyspira sp. Sask30446 gene sequences, purifying any product produced (if of the appropriate size), determining its DNA sequence and comparing that sequence to a reference sequence database for identification. Alternatively, species-specific primers could be easily generated for the other genes (est, glpk, pgm, thi) similar to as developed for Bnox.

Confirmation of identity of cultured isolates could be accomplished through PCR with JH0224/JH0225 (including appropriate positive and negative controls) or PCR from cultured isolates with universal primers or Brachyspira MLST primers followed by DNA sequencing and comparison of the resulting sequence(s) to a reference database.

Molecular diagnostic results are considered in the context of the clinical information.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

Brousseau R, Hill J E, Prefontaine G, Goh S H, Harel J, Hemmingsen S M (2001). Streptococcus suis serotypes characterized by analysis of chaperonin 60 gene sequences. Applied and Environmental Microbiology 67: 4828-4833.
Calderaro A, Bommezzadri S, Piccolo G, Zuelli C, Dettori G, Chezzi C (2005). Rapid isolation of Brachyspira hyodysenteriae and Brachyspira pilosicoli from pigs. Veterinary Microbiology 105: 229-234.
Dorsch M, Stackebrandt E (1992). Some modifications in the procedure of direct sequencing of PCR amplified 16S rDNA. Journal of Microbiological Methods 16: 271-279.
Heinonen, M. et al. 2000. Detectability and prevalence of Brachyspira species in herds rearing health class feeder pigs in Finland. The Veterinary Record, Vol 146, Issue 12, 343-347.
Hill J E, Penny S L, Crowell K G, Goh S H, Hemmingsen S M (2004). cpnDB: a chaperonin sequence database. Genome Research 14: 1669-1675.
Jansen T K, et al. (2010) Vet Pathology. 47:334-338.
Rhode J, et at (2002) J Clin Microbiol. 40(7):2598-2600.
Rasback, T., Jansson, D. S., Johansson, K. E., and Fellstrom, C. (2007a) A novel enteropathogenic, strongly haemolytic spirochaete isolated from pig and mallard, provisionally designated ‘Brachyspira suanatina’ sp. nov. Environ Microbiol 9: 983-991.
Rasback T, Johansson K E, Jansson D S, Fellstrom C, Alikhani M Y, La T et al. (2007b). Development of a multilocus sequence typing scheme for intestinal spirochaetes within the genus Brachyspira. Microbiology 153: 4074-4087.
Thomson, J. R., W. J. Smith and B. P. Murray (1998). “Investigations into field cases of porcine colitis with particular reference to infection with Serpulina pilosicoli.” Veterinary Record 142(10): 235-239.
Thomson, J. R., W. J. Smith, B. P. Murray, et al. (2001). “Porcine enteric spirochete infections in the UK: surveillance data and preliminary investigation of atypical isolates.” Animal Health Research Reviews 2(1): 31-36.

1. A method of screening for or detecting a Brachyspira sp. Sask30446 organism in a sample comprising determining a level of a Brachyspira sp. Sask30446 polynucleotide or polypeptide in the sample, the Brachyspira sp. Sask30446 polynucleotide or polypeptide selected from NADPH oxidase (nox1), chaperonin 60 (cpn60), esterase (est), glucose kinase (glpk), phosphglucomutase (pgm) acetyl-CoA acetyltransferase (thi) and small subunit ribosomal RNA (16S rRNA) wherein detection of the Brachyspira sp. Sask30446 polynucleotide and/or polypeptide is indicative for the presence of the Brachyspira sp. Sask30446 organism in the sample.

2. (canceled)

3. The method of claim 1 for screening for, diagnosing or detecting a Brachyspira sp. Sask30446 infection and/or colitis—unrelated to B. hyodysenteriae and/or B. pilosicoli and/or an increased risk of the colitis, in a subject comprising determining a level of a Brachyspira sp. Sask30446 polynucleotide or polypeptide in a sample of the subject, wherein detection of the Brachyspira sp. Sask30446 polynucleotide and/or polypeptide is indicative that the subject has the infection and/or the colitis related to Brachyspira sp. Sask30446 and/or unrelated to B. hyodysenteriae and/or B. pilosicoli or has an increased risk of developing colitis related to Brachyspira sp. Sask30446 and/or unrelated to B. hyodysenteriae and/or B. pilosicoli.

4.-5. (canceled)

6. The method of claim 1, comprising:
a) detection a NADPH oxidase polynucleotide with at least 92.3% identity to any one of SEQ ID NOs: 7 to 9 or a NADPH polypeptide with at least 92.3% identity to any one of SEQ ID NOs: 11 or 12,
b) detecting a cpn60 polynucleotide with at least 97.5% identity to SEQ ID NO: 25 or a cpn60 polypeptide with at least 98.5% identity to SEQ ID NO: 26;
c) detecting an est polynucleotide with at least 93.5% Identity to SEQ ID NO: 27 or an est polypeptide with at least 95.5% identity to SEQ ID NO: 28;
d) detecting a glpk polynucleotide with at least 95.5% sequence identity to SEQ ID NO: 29 or a glpk polypeptide with at least 99.5% sequence identity to SEQ ID NO: 30:
e) detecting a pgm polynucleotide with at least 93.5% sequence identity to SEQ ID NO: 31 or a pgm polypeptide with at least 99.5% sequence identity to SEQ ID NO: 32;
f) detecting a thi polynucleotide with at least 92.5% sequence identity to SEQ ID NO: 33 or a thi polypeptide with at least 95.5% sequence identity to SEQ ID NO: 34;
g). detecting a 16S rRNA polynucleotide with at least 99.5% sequence identity to SEQ ID NO: 37; and/or
h) detecting a 16S rRNA polynucleotide with at least 99.5% sequence identity to SEQ ID NO: 37 is indicative for the presence of the Brachyspira sp. Sask30446 organism;
wherein detecting the presence of one or more of which is indicative for the presence of the Brachyspira sp. Sask30446 organism.

7.-12. (canceled)

13. The method of claim 3 for screening for, diagnosing or detecting an infection and/or colitis related to Brachyspira sp. Sask30446 and/or unrelated to B. hyodysenteriae and/or B. pilosicoli in a subject comprising:
a. determining a level of a Brachyspira sp. Sask30446 polynucleotide or polypeptide in a sample of the subject;
b. comparing the level of the Brachyspira sp. Sask30446 polynucleotide or polypeptide to a control; and
c. detecting an increased level of the Brachyspira sp. Sask30446 polynucleotide and/or polypeptide relative to the control indicative of the subject having the infection and/or colitis.

14. (canceled)

15. The method of claim 13, wherein the level of the Brachyspira sp. Sask30446 is increased at least 2 fold, at least 3 fold, at least 4 fold, at least 5 fold, at least 6 fold, at least 7 fold, at least 8 fold, at least 9 fold or at least 10 fold relative to the control.

16. The method of claim 13, wherein the determining step comprises:
a. amplifying at least 20 contiguous nucleotides of the NADPDH oxidase polynucleotide, the amplified polynucleotide having greater than 92.3% sequence identity to any one of SEQ ID NO: 7 to 9 or at least 95.5% identity to SEQ ID NO: 11 or 12 when translated;
b. amplifying at least 20 contiguous nucleotides of the cpn60 polynucleotide, the amplified polynucleotide having at least 97.5% identity to SEQ ID NO: 25 or at least 98.5% identity to SEQ ID NO: 26 when translated;
c. amplifying at least 20 contiguous nucleotides of the est polynucleotide, the amplified polynucleotide having at least 93.5% identity to SEQ ID NO: 27 or at least 95.5% identity to SEQ ID NO: 28 when translated:
d. amplifying at least 20 contiguous nucleotides of the glpk polynucleotide, the amplified polynucleotide having at least 95.5% sequence identity to SEQ ID NO: 29 or at least 99.5% sequence identity to SEQ ID NO: 30 when translated;
e. amplifying at least 20 contiguous nucleotides of the pgm polynucleotide, the amplified polynucleotide having with at least 93.5% sequence identity to SEQ ID NO: 31 at least 99.5% sequence identity to SEQ ID NO: 32 when translated;
f. amplifying at least 20 contiguous nucleotides of the thi polynucleotide, the amplified polynucleotide having at least 92.5% sequence identity to SEQ ID NO: 33 or at least 95.5% sequence identity to SEQ ID NO: 34 when translated; and/or
g. amplifying at least 20 contiguous nucleotides of the 16S rRNA polynucleotide the amplified polynucleotide having at least 99.5% sequence identity to SEQ ID NO: 37;
optionally, wherein at least 40, at least 60, at least 80, at least 100, at least 120, at least 140, at least 160, at least 180, or at least 200 contiguous nucleotides are amplified or at least 200-250, 250-300, 300-350, 350-400 or more contiguous nucleotides are amplified.

17.-23. (canceled)

24. The method of claim 16, wherein the presence and/or level of Brachyspira sp. Sask30446 polynucleotide is determined using an amplification reaction such as PCR, RT-PCR, optionally qPCR or qRT-PCR, employing an oligonucleotide primer or primer pair that hybridizes to the Brachyspira sp. Sask30446 polynucleotide or complement thereof.

25. The method of claim 24, wherein the Brachyspira sp. Sask30446 polynucleotide is:
a. NADPH oxidase and the level is determined using a primer selected from SEQ ID NO:34; SEQ ID NO: 5 and 6, or a primer pair selected from SEQ ID NO:3 and 4; and SEQ ID NO: 5 and 6;
b. cpn60 and the level is determined using a primer selected from SEQ ID NOs: 23 and 24 or a primer pair consisting SEQ ID NOs: 23 and 24;
c. est and the level is determined using a primer selected from SEQ ID NO:15 and 16 or a primer pair consisting of SEQ NO: 15 and 16:
d. glpk and the level is determined using a primer selected from SEQ ID NO:17 and 18 or a Primer pair consisting of SEQ ID NO:17 and 18;
e. pgm and the level is determined using a primer selected from SEQ ID NO:19 and 20 or a primer pair consisting of SEQ ID NO: 19 and 29;
f. thi and the level is determined using a primer selected from SEQ ID NO:21 and 22 or a primer pair consisting of SEQ ID NO:21 and 22; and/or
g. 16S rRNA and the level is determined using a primer selected from SEQ ID NO:35 and 36 or a primer pair consisting of SEQ ID NO:35 and 36.

26.-31. (canceled)

32. The method of claim 24, wherein the amplifying uses one or more primers selected from SEQ ID NO: 1, 2, and a species-specific primer.

33. The method of claim 16, wherein the method further comprises sequencing the amplified polynucleotide and comparing the sequence to a reference sequence.

34. The method of claim 13 wherein the determining step comprises;
a. amplifying at least 20 contiguous nucleotides of a Brachyspira sp. Sask30446 polynucleotide, using a primer or primer set optionally selected from SEQ ID NO:1, 2 and/or a species specific primer;
b. hybridizing the amplified sequence with a polynucleotide probe which specifically binds the Brachyspira sp. Sask30446 polynucleotide or sequencing the amplified sequence and comparing to a reference sequence.

35.-37. (canceled)

38. The method of claim 1, wherein the level of the Brachyspira polynucleotide or polypeptide is determined by:
a. contacting the sample with an analyte specific binding agent (ASBA) which specifically binds the Brachyspira sp. Sask30446 polypeptide or the Brachyspira sp. Sask30446 polynucleotide; and
b. detecting the level of the Brachyspira sp. Sask30446 polypeptide and/or polynucleotide in the sample.

39.-40. (canceled)

41. The method of claim 38, wherein the ASBA is:
a. a polynucleotide primer or probe and comprises at least 30, at least 40, at least 50, at least 60, at least 70, at least 80, at least 90, at least 100 or more nucleotides of any one of SEQ ID NOs: 7 to 9, 25, 27, 29, 31, 35 and 37; or
b. an antibody specific for a polypeptide comprising SEQ ID NOs: 11, 12, 26, 28, 30, 32, 34 or 36 or encoded by any one of SEQ ID NOs: 7 to 9, 25, 27, 29, 31, 35 and 37.

42. (canceled)

43. The method of claim 3, wherein the colitis is a mucohaemorrhagic colitis associated with bloody diarrhea.

44. The method of claim 1, wherein the sample comprises cells or tissue of duodenum, ileum, jejunum, caecum, rectum or colon, for example colon cells, colon contents (e.g. fecal material) and/or gastrointestinal content.

45. (canceled)

46. The method of claim 1, wherein the subject is a pig.

47.-49. (canceled)

50. A composition comprising;
a. an isolated polynucleotide comprising:
i. at least 15, at least 20, at least 30, at least 40, at least 50, at least 100, at least 150, at least 200., at least 300, at least 400, at least 500, at least 600, at least 700 or at least 800 nucleotides contiguous nucleotides of anyone of SEQ ID NOs: 3-9, 25, 27, 29, 31, 35 and 37;
ii. a sequence at least 92.5% identical to any one of SEQ ID NOs: 3-9, 25, 37, 29, 31, 33.35 and 37; and/or
iii. a complement of i) or ii);
b. an isolated polypeptide comprising at least 10, at least 20, at least 30, at least 40, at least 50, at least 60, at least 70, at least 80, at least 90 or at least 100 contiguous amino acids of any one of SEQ ID NOs:11, 12, 26, 28, 30, 32, 34 or 37 or at least 10 contiguous amino acids encoded by any one of SEQ ID NOs: 7 to 9, 25, 27, 29, 31, 33, 35 and 37; or
c. an isolated antibody specific for a polypeptide of b); and
optionally a suitable carrier.

51. The composition of claim 50 comprising at least 2 isolated polynucleotides, at least 2 polypeptides or at least 2 antibodies.

52. A kit comprising the composition of claim 50 and one or more additional components selected from a polynucleotide useful as a positive or negative control; an analyte specific binding agent specific for a Brachyspira sp. Sask30446—polynucleotide or polypeptide; a standard and a reaction tube or plate.

53.-54. (canceled)

55. The method of claim 1, further comprising culturing the sample, for example to obtain a colony, prior to determining a level of a Brachyspira sp. Sask30446 polynucleotide or polypeptide.

56. (canceled)


SEQUENCE LISTING (37 sequences)

SEQ ID NO: 1 - DNA, 23 bp, Artificial sequence
  Synthetic construct
tagcytgcggtatygcwctttgg

SEQ ID NO: 2 - DNA, 23 bp, Artificial Sequence
  Synthetic construct
cttcagaccayccagtagaagcc

SEQ ID NO: 3 - DNA, 23 bp, Brachyspira sp. Sask 30446
tcgctaaattattccaacaagga

SEQ ID NO: 4 - DNA, 21 bp, Brachyspira sp. Sask 30446
aaacgcatttctattccagca

SEQ ID NO: 5 - DNA, 24 bp, Brachyspira sp. Sask 30446
tcatagatgctggaatagaaatgc

SEQ ID NO: 6 - DNA, 22 bp, Brachyspira sp. Sask 30446
gcaccgttaggtaaagtttcca

SEQ ID NO: 7 - DNA, 241 bp, Brachyspira sp. Sask 30446
tcgctaaatttatccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgttt

SEQ ID NO: 8 - DNA, 241 bp, Brachyspira sp. Sask 30446
tcgctaaattattccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgttt

SEQ ID NO: 9 - DNA, 810 bp, Brachyspira sp. Sask 30446
cctgaaggtttgaaatctgaaggcatcgatgtttatatgggacatgaagtaacaaaaatagactgggctaacaaaaaattacatatcaaagaattaaaaacaggtaaagagtttgaagacaattatgataaacttatacttgctactggttcttggcctgtaactcctcctatagaaggattaaaacaagaaggtactgaatacggtcttaaaaaaggtattttcttcgctaaattattccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgtttaggtgaaactgttaaaaagtttgaaggtgatgacagagttaaaaaagttgttaccgataaaggttcttatgatgtagatatggtagttatgtctgttggtttcagacctaatagcgaactttataaagactatttggaaactttacctaacggtgctattgtagtagatactactatgagatcaagcaaagatcctgatgtttatgctataggagactgtgctactgtatattcaagagcttctgaaaaacaagagtatattgctttagctactaatgctgtaagaatgggtattgttgctgctaataatgctttaggaaaacatgttgaatactgcggtact

SEQ ID NO: 10 - DNA, 809 bp, Brachyspira innocens
cctgaaagtttaaaagctgaaggcatagatgtttatatgggacatgatgtaacaaaaatagactgggctaataaaaaattacatgttaaagaattaaaaacaggtaaagagtttgatgataattatgacaaacttattcttgctacaggttcttggcctgtaactcctcctatagaaggtttaatgcaggaaggtactgaatacggacttaaaaaaggtattttcttctctaaattattccagcaaggacaagaaattattgatgaaatagctaaacctgaagtaaaaaaagttatggtagttggtgctggttatataggtgttgaacttatagaagctttcaaaaatcatggtaaagaagttatattaatggaagctatgcctagagttatggctaactattttgacaaagaaattactgatgaagctgaaaaaagaatcaaagaagctggcatagaaatgcatttaggtgaaactgttaaaaaatttgaaggtgatgacagagttaaaaaagttattactgacaaaggttcttatgatgtagatatggtagttatgtctgttggtttcagacctaatagtgagctttataaagattatttagaaactttacctaatggtgctataaaagtagacactactatgaaaactacaaaagatcctaatgtatttgctataggagactgtgctactgtatattcaagagcttctggaaaagaagaatacatcgctttagctactaatgctgtaagaatgggattgttgctgctaacaatgctttaggaaaacatgttgaatactgcggtact

SEQ ID NO: 11 - protein, 79 aa, Brachyspira sp. Sask 30446
AKFIQQGQDIINEIAKPEVKKVMVVGAGYIGVELIEAFKNHGKEVILMESMPRVMANYFDKEITDEAEKRIIDAGIEMR

SEQ ID NO: 12 - protein, 270 aa, Brachyspira sp. Sask 30446
PEGLKSEGIDVYMGHEVTKIDWANKKLHIKELKTGKEFEDNYDKLILATGSWPVTPPIEGLKQEGTEYGLKKGIFFAKLFQQGQDIINEIAKPEVKKVMVVGAGYIGVELIEAFKNHGKEVILMESMPRVMANYFDKEITDEAEKRIIDAGIEMRLGETVKKFEGDDRVKKVVTDKGSYDVDMVVMSVGFRPNSELYKDYLETLPNGAIVVDTTMRSSKDPDVYAIGDCATVYSRASEKQEYIALATNAVRMGIVAANNALGKHVEYCGT

SEQ ID NO: 13 - protein, 270 aa, Brachyspira murdochii
PESLKAEGIDVYMGHDVTKIDWANKKLHVKELKTGKEFDDNYDKLILATGSWPVTPPIEGLMQEGTEYGLKKGIFFSKLFQQGQEIIDEIAKPEVKKVMVVGAGYIGVELIEAFKNHGKEVILMEAMPRVMANYFDKEITDEAEKRIKEAGIEMHLGETVKKFEGDDRVKRVVTDKGSYDVDMVVMSVGFRPNSELYKDYLETLPNGAIKVDTTMKTTKDPNVFAIGDCATVYSRASGKEEYIALATNAVRMGIVAANNALGKHVEYCGT

SEQ ID NO: 14 - protein, 79 aa, Brachyspira pilosicoli
SKLYQQGQDIINEIAKPEVKKVMVVGAGYIGVELIEAFKNHGKEVILMEAMPRVMANYFDKEITDEAEKRIKDAGIELH

SEQ ID NO: 15 - DNA, 21 bp, Artificial Sequence
  Synthetic construct
gatgcttcaggcggagttatg

SEQ ID NO: 16 - DNA, 23 bp, Artificial Sequence
  Synthetic construct
ccacactcatagcataaatactg

SEQ ID NO: 17 - DNA, 21 bp, Artificial Sequence
  Synthetic construct
aggctgggtagaacataatgc

SEQ ID NO: 18 - DNA, 23 bp, Artificial Sequence
  Synthetic construct
tctttactttgataagcaatagc

SEQ ID NO: 19 - DNA, 22 bp, Artificial Sequence
  Synthetic construct
gttggtactaacagaatgaata

SEQ ID NO: 20 - DNA, 20 bp, Artificial Sequence
  Synthetic construct
ccgtctttatcgcgtacatt

SEQ ID NO: 21 - DNA, 22 bp, Artificial Sequence
  Synthetic construct
tgtgttatacaatcagcacttc

SEQ ID NO: 22 - DNA, 22 bp, Artificial Sequence
  Synthetic construct
gtagtaagtattctagctccag

SEQ ID NO: 23 - DNA, 50 bp, Artificial Sequence
  Synthetic construct
cgccagggttttcccagtcacgacgannnngcnggngayggnacnacnac

SEQ ID NO: 24 - DNA, 50 bp, Artificial Sequence
  Synthetic construct
agcggataacaatttcacacaggayknykntcnccraanccnggngcytt

SEQ ID NO: 25 - DNA, 555 bp, Brachyspira sp. Sask 30446
gctactgttttagctcaggctatggttaaagaaggtttgaaaaatgtaactagcggtgctaatcctatgcttattaaaagaggtatagaaaaagctgttagcgaaatagttgcttatatcaaatctgaagctaaacaaattaaaggcaaagaagaaattgctcaggttgctactatttctgctaacaatgataaagaaattggtgctttaatcagtgatgctatggaaaaagttggtaaagaaggcgttatcactgtagaagaagctaaatctttagaaactagtctttctttggtagaaggtatgcagtttgacagaggttatatctctccatatttcgtaactaacggagatagtatgactgctgaattagaagatgctttacttcttatctatgataaaaaaatctctaacatgaaagaacttcttcctatacttgaaaaaattgctcagactggaagacctttcattattatcgctgaagatattgaaaatgaagctttggctactttggtacttaacaaaatgagaggagtattaaatgtatgtgctgtt

SEQ ID NO: 26 - protein, 185 aa, Brachyspira sp. Sask 30446
ATVLAQAMVKEGLKNVTSGANPMLIKRGIEKAVSEIVAYIKSEAKQIKGKEEIAQVATISANNDKEIGALISDAMEKVGKEGVITVEEAKSLETSLSLVEGMQFDRGYISPYFVTNGDSMTAELEDALLLIYDKKISNMKELLPILEKIAQTGRPFIIIAEDIENEALATLVLNKMRGVLNVCAV

SEQ ID NO: 27 - DNA, 581 bp, Brachyspira sp. Sask 30446
aaatatttagaagagagaaatataggttttgatgttggagttacaaaggttcctttggtttgtcagtcttgtatatttgatttgcgtgttggggattataaagttcgtccggatattaatatggcttatgaggcttgtatcaatgctcaaaataataatccaaaaatgggaaattatggagctggtacaggtgctagtgttggaaaaatacttggtgctgattatactatgaagtctgggcttggtttttatgctgttcaaattgatgatgttaaggttggtgctatagttgctcttaatgctttcggcgatatttatgattatgataatggaaaaatgattgctggtcttcttaatgaaaataaagatgggtttagaagttctgaagaggagcttataaaaataacgcagaataataatttgtcttttacttctaataataatatagttacaaatactacaataggagctgtaattacaaatgccaagtttacaaagtcacaaatgggaaaaatagcttctatggcacataacggctttgctagggctataaaaccagtgcatactacagtagatggcga

SEQ ID NO: 28 - protein, 193 aa, Brachyspira sp. Sask 30446
KYLEERNIGFDVGVTKVPLVCQSCIFDLRVGDYKVRPDINMAYEACINAQNNNPKMGNYGAGTGASVGKILGADYTMKSGLGFYAVQIDDVKVGAIVALNAFGDIYDYDNGKMIAGLLNENKDGFRSSEEELIKITQNNNLSFTSNNNIVTNTTIGAVITNAKFTKSQMGKIASMAHNGFARAIKPVHTTVDG

SEQ ID NO: 29 - DNA, 930 bp, Brachyspira sp. Sask 30446
ggagtttgcaggagctatacagattgctggagttaagcctgaagagatagctgctattggtattacaaatcaaagggaaactactgttgtatgggataaaaatactggagagcctatttataatgctatagtatggcaatgtagaagaactgctcctatttgcgatgatttaaagaaaaaaggacttgatacttatataagagaaaatacaggtttagttgttgatgcttatttttctggtacaaaaataaaatggatacttgataatgttcctggtgctagagaaaaagccaataaaggagaattactatttggtacaatagacacttggcttgtatggaaacttacaggcggtaaagttcatgttacagactatactaatgcatcaaggactatgatttataatatcaaagatttaaaatgggacgaaaacattttaagagaattagacattcctatgagcatgcttccagaggtaaaagattcttcttgtgtttacggatatgctaatattaacggtaaagaagtacctatatctggaatagcaggagaccagcagtcagcattattcggtcaggctggatttaataagggcgacacaaaaaatacttatggtactggaagtttcattcttatgaatataggagagaattttatattaagtaaaaacggacttattactactataggtataggatataatggaaaaatagaatatgctttggaaggttctgtatttatagctggtgctgttatacaatgggtacgcgatgaattaagacttcttcatgatgcaaaagacactgaatatttcgctactaaagtaaaagatactaacggagtttatttagtacctgcatttgttggtcttggtgctccttattgggatatgtatgctagaggctgtttagtaggtattacaagaggggt

SEQ ID NO: 30 - protein, 310 aa, Brachyspira sp. Sask 30446
EFAGAIQIAGVKPEEIAAIGITNQRETTVVWDKNTGEPIYNAIVWQCRRTAPICDDLKKKGLDTYIRENTGLVVDAYFSGTKIKWILDNVPGAREKANKGELLFGTIDTWLVWKLTGGKVHVTDYTNASRTMIYNIKDLKWDENILRELDIPMSMLPEVKDSSCVYGYANINGKEVPISGIAGDQQSALFGQAGFNKGDTKNTYGTGSFILMNIGENFILSKNGLITTIGIGYNGKIEYALEGSVFIAGAVIQWVRDELRLLHDAKDTEYFATKVKDTNGVYLVPAFVGLGAPYWDMYARGCLVGITRGV

SEQ ID NO: 31 - DNA, 945 bp, Brachyspira sp. Sask 30446
caggtttagctaattatatattaaaacaaggcggaagtgattataaagttgctataggatatgattcaagaaataattctgatgtattttcaaaagctgctgctgagatactttcttcaaacggaataaaagtttatttatatgatgatattcaccctatttcacttctttcttatgctgttagaagtttaggctgtattgcaggaatagttgttactgctagtcataaccctaaggaatataatggatataaagtttattggactgacggagctcaggttataccgcctcatgacaaaaatatcatagatgaagtattaaaagttaagcctgaagaagtaaaaatgggcgactcttcaaaaattactataataggaaaagatattgaagataaatacatgaatgatttgatggtatatttagttaatccagacatcattaaaaaacatcatgatataaaaatagtttataccccaattcatggttctggatacaaaatggttcctatggctttaagaaaggctggttttacaaatttaacaacattagaaggtgctcagcctccagacggaaattttcctactgtagaatctcctaacccagaaaatcctgaagcattgcagatagccgttaataaagctaaagagataggtgcagaacttgttatgggtaccgacccagactgcgacagaatgggatgtgctttgcttactaaagatggaagctatatgtatcttacaggtaatcagataggatccataatggcatactatctcatcacaaacaaaaaaaatattaaaaatccgtacatagtaaaaacaatagtaacaactgaactagctagggctattgctgatgctaataatgttaagatttacgatgtacttacaggttttaaatggattgctgatgttatagaaagagataaagaaggtacatatttat

SEQ ID NO: 32 - protein, 314 aa, Brachyspira sp. Sask 30446
GLANYILKQGGSDYKVAIGYDSRNNSDVFSKAAAEILSSNGIKVYLYDDIHPISLLSYAVRSLGCIAGIVVTASHNPKEYNGYKVYWTDGAQVIPPHDKNIIDEVLKVKPEEVKMGDSSKITIIGKDIEDKYMNDLMVYLVNPDIIKKHHDIKIVYTPIHGSGYKMVPMALRKAGFTNLTTLEGAQPPDGNFPTVESPNPENPEALQIAVNKAKEIGAELVMGTDPDCDRMGCALLTKDGSYMYLTGNQIGSIMAYYLITNKKNIKNPYIVKTIVTTELARAIADANNVKIYDVLTGFKWIADVIERDKEGTYL

SEQ ID NO: 33 - DNA, 821 bp, Brachyspira sp. Sask 30446
tattaatgccggaatacctgtagacaaaccggcattaactataaatatattatgcggttcaggattaagagctgtatcaatggcagcacaaatgattaaatcaggagatgctgatatagtagttgcaggcggtactgaaaatatgagtatggctccatatacttcttcagctatgagaatgggtgctagaatgggcgaaagtaaaatgcaggatactttacttaatgatgctcttatttgtgcttttgagcattatcatatgggagttactgctgaaaatgttgcagaacaatggggtattacaagacaagaacaagatgagtttgcatgcagaagccagaacagagcagaagaagcagtaaaaagcggaagatttaaagatgaaatagtacctgttacaatcaaaactagaaaaggcgaaatagtagttgatacagatgaacaccctacttttggtactactatggaatctttagctaaattaaaacctgcattcaaaaaagatggtactgttactgctggtaatgcttctggtatcaatgatgctgcttcagctgtagttattatgtctaaagaaaaagctgatgagcttggaattaaacctatggctaagattttaggttatgctactcatggtgttgagcctagaataatgggtataggacctatagaggcaactagaaaggctttgaaaatggctaatcttacagttgaagatatggacttaatagaaagtaatgaggctttcgctgctcagtctatagctgttgcccgtgagttaaaattcaatatggacatagttaatgtcaatggcggagcta

SEQ ID NO: 34 - protein, 273 aa, Brachyspira sp. Sask 30446
INAGIPVDKPALTINILCGSGLRAVSMAAQMIKSGDADIVVAGGTENMSMAPYTSSAMRMGARMGESKMQDTLLNDALICAFEHYHMGVTAENVAEQWGITRQEQDEFACRSQNRAEEAVKSGRFKDEIVPVTIKTRKGEIVVDTDEHPTFGTTMESLAKLKPAFKKDGTVTAGNASGINDAASAVVIMSKEKADELGIKPMAKILGYATHGVEPRIMGIGPIEATRKALKMANLTVEDMDLIESNEAFAAQSIAVARELKFNMDIVNVNGGA

SEQ ID NO: 35 - DNA, 19 bp, Artificial Sequence
  Synthetic construct
gagtttgatcctggctcag

SEQ ID NO: 36 - DNA, 18 bp, Artificial Sequecne
  Synthetic construct
gwattaccgcggckgctg

SEQ ID NO: 37 - DNA, 464 bp, Brachyspira sp. Sask 30446
agcgaacgttggcgatgcgtcttaagcatgcaagtcgagcggacttattcgggcaactggataagttagcggcgaactggtgagtaacacgtaggtaatctgccgtagagtgggggataacccatggaaacatggactaataccgcatatactcttactacacaagtagagtagaggaaaggagcaatccgctttacgatgagcctgcggcctattagcctgttggtggggtaacggcctaccaaagctacgataggtagccgacctgagagggtgaccggccacattgggactgagatacggcccagactcctacgggaggcagcagctgagaatcttccacaatggacgaaagtctgatggagcgacatcgcgtgagggatgaaggccttcgggttgtaaacctcggaaattatcgaagaatgagtgacagtagataatgtaagcctcggctaactacgtgc